SEQUENCE LISTING (16 sequences)

SEQ ID NO: 1 - DNA, 1264 bp, Homo sapiens
  misc_feature (1)..(1264)
ctggctcggttaaaaagctggttgcgtagattcctgtcaatgctcaggatcctctgccttgtgatatctggagataagtcaacgccttgcaggacgcttacatgctcgggcagtacctctctcagcaacacctccatgcactggtatacaaagtccccctcaccccagccgcgacccttcaaggccaagaggcggcagagcccgaggcctgcacgagcagctctctcttcaggagtgaaggaggccacgggcaagtcgccctgacgcagacgctccaccagggccgcgcgctcgccgtccgccacataccgctcgtagtattcgtgctcagcctcgtagtggcgcctgacgtcgcgttcgcgggtagctacgatgaggcggcgacagaccaggcacagggccccatcgccctccggaggctccaccaccaaataacgctgggtccactcgggccggaaaactagagcctcgtcgacttccatcttgcttcttttgggcgtcatccacattctgcgggaggccacaagagcagggccaacgttagaaaggccgcaaggggagaggaggagcctgagaagcgccaagcacctcctccgctctgcgccagatcacctcagcagaggcacacaagcccggttccggcatctctgctcctattggctggatatttcgtattccccgagctcctaaaaacgaaccaataggaagagcggacagcgatctctaacgcgcaagcgcatatccttctaggtagcgggcagtagccgcttcagggagggacgaagagacccagcaacccacagagttgagaaatttgactggcattcaagctgtccaatcaatagctgccgctgaagggtggggctggatggcgtaagctacagctgaaggaagaacgtgagcacgaggcactgaggtgattggctgaaggcacttccgttgagcatctagacgtttccttggctcttctggcgccaaaatgtcgttcgtggcaggggttattcggcggctggacgagacagtggtgaaccgcatcgcggcgggggaagttatccagcggccagctaatgctatcaaagagatgattgagaactggtacggagggagtcgagccgggctcacttaagggctacgacttaacgggccgcgtcactcaatggcgcggacacgcctctttgcccgggcagaggcatgtacagcgcatgcccacaacggcggaggccgccgggttccctgacgtgccagtcaggccttctccttttcc

SEQ ID NO: 2 - DNA, 24 bp, Artificial Sequence
  RTPCR-MLH1 F primer
cggttaactacccaatgcctcaac

SEQ ID NO: 3 - DNA, 23 bp, Artificial Sequence
  RTPCR-MLH1 R primer
ttctcgactaacagcatttccaa

SEQ ID NO: 4 - DNA, 22 bp, Artificial Sequence
  RTPCR-GAPDH F primer
ccaggtggtctcctctgacttc

SEQ ID NO: 5 - DNA, 24 bp, Artificial Sequence
  RTPCR-GAPDH R primer
tcataccaggaaatgagcttgaca

SEQ ID NO: 6 - DNA, 20 bp, Artificial Sequence
  Genomic PCR-MLH1 (-780 to +483) F primer
ctggctcggttaaaaagctg

SEQ ID NO: 7 - DNA, 20 bp, Artificial Sequence
  Genomic PCR-MLH1 (-780 to +483) R primer
ggaaaaggagaaggcctgac

SEQ ID NO: 8 - DNA, 29 bp, Artificial Sequence
  Forward primer for amplification of -27, -16 and -1 region
ggtatttttgtttttattggttggatatt

SEQ ID NO: 9 - DNA, 27 bp, Artificial Sequence
  Reverse primer for amplification of -27, -16 and -1 region
aataccaatcaaatttctcaactctat

SEQ ID NO: 10 - DNA, 22 bp, Artificial Sequence
  Pyrosequence primer for -27, -16 and +1
gtaagagtatatttttttaggt

SEQ ID NO: 11 - DNA, 29 bp, Artificial Sequence
  Forward primer for amplification of -70 and -63 region
ggtatttttgtttttattggttggatatt

SEQ ID NO: 12 - DNA, 27 bp, Artificial Sequence
  Reverse primer for amplification of -70 and -63 region
aataccaatcaaatttctcaactctat

SEQ ID NO: 13 - DNA, 25 bp, Artificial Sequence
  Pyrosequence primer for -70 and -63
ttttaaaaaagaattaataggaaga

SEQ ID NO: 14 - DNA, 22 bp, Artificial Sequence
  Forward primer for amplification of +157 and +172 region
aggtgattggttgaaggtattt

SEQ ID NO: 15 - DNA, 29 bp, Artificial Sequence
  Reverse primer for amplification of +157 and +172 region
ccaattctcaatcatctctttaataacat

SEQ ID NO: 16 - DNA, 19 bp, Artificial Sequence
  Pyrosequence primer for +157 and +172 region
gattggttgaaggtatttt

(19) United States
(12) Patent Application Publication
(10) Pub. No.: US 2021/0106612 A1
(43) Pub. Date: Apr. 15, 2021

(54) TARGET GENE TRANSCRIPTIONAL REPRESSION INHIBITOR THAT CAN BE DESIGNED IN A SEQUENCE-SPECIFIC MANNER, COMPOSITION CONTAINING SAME, AND USE THEREOF

(71) Applicant: National University Corporation Chiba University, Chiba-shi (JP)

(72) Inventors: Atsushi KANEDA, Chiba (JP); Ken-ichi SHINOHARA, Chiba (JP); Tetsuhiro NEMOTO, Chiba (JP)

(73) Assignee: National University Corporation Chiba University, cjiba-shi (JP)

(21) Appl. No.: 16/074,258

(22) PCT Filed: Feb. 1, 2017
(86) PCT No.: PCT/JP2017/003623

§ 371 Date: Oct. 23, 2018

(30) Foreign Application Priority Data
Feb. 1, 2016 (JP) 2016-016951

(51) Int. Cl. A61K 31/787; A61K 31/165

(52) U.S. Cl. CPC A61K 31/787; A61K 31/165

(57) ABSTRACT

The present invention provides a target gene transcriptional repression inhibitor that can be designed in a sequence-specific manner, a composition containing the same, and use thereof. More specifically, the present invention provides a composition for use in inhibiting repression of gene expression by DNA methylation in a living subject, comprising pyrrole imidazole polyamide or a conjugate of pyrrole imidazole polyamide and a histone modifying enzyme inhibitor, wherein the pyrrole imidazole polyamide or the conjugate of pyrrole imidazole polyamide and a histone modifying enzyme inhibitor is designed so as to bind in a sequence-specific manner to a minor groove of promoter region DNA of the gene.

### REFERENCE OF RELATED APPLICATION

The present application claims the priority based on Japanese Patent Application No. 2016-016951 (filing date: Feb. 1, 2016), the contents of which are incorporated herein by reference in their entirety.

### TECHNICAL FIELD

The present invention relates to a target gene transcriptional repression inhibitor that can be designed in a sequence-specific manner, a composition containing the same, and use thereof.

### BACKGROUND ART

Abnormal hypermethylation of DNA causes transcriptional repression of a gene, particularly, when occurring in a promoter region serving as an expression regulatory site of the gene. Further, abnormal hypermethylation of DNA in a promoter region of a cancer suppressor gene is an important cause of carcinogenesis for many cancers such as large intestine cancer.

In therapeutic strategies for cancers, an approach of reducing methylation of a hypermethylated gene region is adopted in order to improve the expression of a cancer suppressor gene. For example, 5-aza-2′-deoxycytidine and 5-azacytidine are also known as anticancer agents that are administered to function as DNA demethylating agents. However, these DNA demethylating agents nonspecifically demethylate the whole genomic DNA in living subjects (Non Patent Literatures 1 and 2). Therefore, adverse reactions caused thereby cannot be avoided.

Pyrrole imidazole polyamide is a DNA binding molecule that can be designed so as to bind in a sequence-specific manner to DNA (Non Patent Literature 3). Various approaches of engineering epigenetic modification in a sequence-specific manner using a fusion drug of pyrrole imidazole polyamide and a DNA demethylating agent or a histone deacetylase inhibitor have started to be reported (Non Patent Literature 4). Also, an approach of inhibiting methylation of plasmid DNA in a sequence-specific manner using pyrrole imidazole polyamide has been reported (Non Patent Literature 5). Non Patent Literature 5 has concluded that pyrrole imidazole polyamide directly inhibits DNA methylation by competing with the binding of DNA methyltransferase (DNMT), and conceptually presents the competition of pyrrole imidazole polyamide with DNA methyltransferase. Non Patent Literature 5 has merely verified that pyrrole imidazole polyamide can inhibit methylation of a C residue present within a binding sequence of several bases long to which the pyrrole imidazole polyamide binds. In consideration of the competition with DNMT proposed as a mechanism, it is predicted that this has no influence on C residues in the neighborhood of this sequence. On the other hand, gene repression is attributed to hypermethylation throughout a promoter region. According to Non Patent Literature 5, therefore, pyrrole imidazole polyamide can merely have no or very slight influence on repression of gene expression, and the pyrrole imidazole polyamide alone is obviously unsuitable for causing the inhibition of repression of gene expression in living subjects.

### CITATION LIST

### Non Patent Literature

Non Patent Literature 1: Patai A V et al., PLoS One, 20, 10 (8): e0133836, 2015
Non Patent Literature 2: Taylor S M et al., Cell, 17 (4): 771-779, 1979
Non Patent Literature 3: White, S et al., Nature, 391, 468, 1998
Non Patent Literature 4: Pandian G N et al., Sci. Rep., 24 (4), 3843, 2014
Non Patent Literature 5: JeenJoo S K et al., JACS, 136, 3687-3694, 2015

### SUMMARY OF INVENTION

### Technical Problem

The present invention provides a target gene transcriptional repression inhibitor that can be designed in a sequence-specific manner, a composition containing the same, and use thereof.

### Solution to Problem

The present inventors have found that pyrrole imidazole polyamide (PIP) can be enriched and distributed in the nucleus in a cell, can suppress methylation of a target site in a cell, and is capable of suppressing methylation of a DNA region in the neighborhood of the target site. The inventors have particularly found that four monomer units of PIP suffice for suppressing the methylation of a DNA region in the neighborhood of the target site. The present inventors have also revealed that this can inhibit transcriptional repression of the gene by methylation. The present inventors have further found that the transcriptional repression of the gene can be effectively inhibited by pyrrole imidazole polyamide treatment after DNA demethylation treatment and, preferably, inhibition treatment of histone deacetylation. Furthermore, the present inventors have found that the conjugation of a compound such as a histone modifying enzyme inhibitor to pyrrole imidazole polyamide allows the compound to be delivered to genomic DNA in a sequence-dependent manner. The present invention is an invention made on the basis of these findings.

Specifically, the present invention provides the following aspects.
(1) A composition for use in inhibiting repression of gene expression by DNA methylation in a living subject, comprising pyrrole imidazole polyamide, wherein the pyrrole imidazole polyamide is designed so as to bind in a sequence-specific manner to a minor groove of DNA in the promoter region of the gene.
(2) A composition for use in inhibiting repression of gene expression by DNA methylation in a living subject, comprising a conjugate of pyrrole imidazole polyamide and a histone modifying enzyme inhibitor, wherein the pyrrole imidazole polyamide in the conjugate is designed so as to bind in a sequence-specific manner to a minor groove of DNA in the promoter region of the gene.
(3) The composition according to (1) or (2), wherein the DNA methylation is DNA methylation that occurs intracellularly after DNA demethylation treatment.
(4) The composition according to (3), wherein the DNA methylation is DNA methylation that occurs intracellularly after DNA demethylation treatment and inhibition treatment of histone deacetylation.
(5) The composition according to (4) or (4), wherein the DNA demethylation treatment is to be performed by 5-aza-2′-deoxycytidine (AZA) treatment.
(6) The composition according to (4) or (5), wherein the inhibition treatment of histone deacetylation is to be performed by trichostatin A treatment.
(7) The composition according to any of (1) to (6), wherein the gene is a cancer suppressor gene.
(8) The composition according to (7), wherein the gene is MLH1 gene.
(9) A pharmaceutical composition for treating a cancer in a subject having the cancer, comprising a composition according to (7) or (8), whereby the pharmaceutical composition is capable of inhibiting transcriptional repression of a cancer suppressor gene.
(10) The pharmaceutical composition according to (9), wherein the cancer is a cancer selected from large intestine cancer, stomach cancer, gastric pit-type stomach cancer, esophageal cancer, head and neck squamous cell carcinoma, non-small cell lung cancer and colorectal cancer, and the cancer suppressor gene having methylated DNA in the promoter region is MLH1 gene.
(11) The pharmaceutical composition according to (9) or (10), wherein the pharmaceutical composition is to be used in combination with a DNA demethylating agent and a histone deacetylation inhibitor.
(12) A pharmaceutical composition for use in preventing the onset of a cancer in a subject with increased methylation of MLH1 gene, or for use in reducing a risk of developing a cancer in the subject, comprising a composition according to (8).
(13) A DNA methylation inhibitor for use in inhibiting DNA methylation in target DNA or the vicinity thereof in a living subject, comprising pyrrole imidazole polyamide, wherein the pyrrole imidazole polyamide is designed so as to bind in a sequence-specific manner to the target DNA.

### BRIEF DESCRIPTION OF DRAWINGS

FIG. 1 is a diagram showing that pyrrole imidazole polyamide (PIP1) binding in a sequence-specific manner to a region from −19 to −13 of the promoter region of the MLH1 gene was designed as one example. In the diagram, TSS denotes a transcription start site (transcription start point).

FIG. 2 shows the in vitro binding properties (FIG. 2A) and intracellular distribution (FIG. 2B) of the designed PIP1.

FIG. 3 shows a plasmid harboring an integrated DNA region containing the promoter region of the MLH1 gene (FIG. 3A), a relative position of a site at which a methylation level was confirmed on the DNA region (FIG. 3B), and the suppression of a DNA methylation level by PIP1 (FIG. 3C).

FIG. 4A shows the methylation level of the promoter region of the MLH1 gene in cells treated with 5-aza-2′-deoxycytidine (AZA) or 5-aza-2′-deoxycytidine and trichostatin A (AZA+TSA). FIG. 4B shows change in the expression level of the MLH1 gene by the treatment described above.

FIG. 5 shows change in the methylation level of the region described above (FIG. 5A) and change in the expression level of the MLH1 gene (FIG. 5B) in cells cultured under hypoxic conditions after the treatment described above.

FIG. 6 shows that PIP1 can inhibit a rise in methylation level (FIGS. 6A and 6B), can cancel repression of MLH1 gene expression (FIGS. 6C and 6D), and is effective for suppressing methylation even at a site of the promoter region distant from the binding site of PIP1 (FIG. 6E).

FIG. 7 shows a schematic diagram of four-base recognition-type PIPs. These PIPs form a homodimer on a DNA sequence.

FIG. 8 shows results of genome-wide methylation analysis using Im0 or Im3 and results of methylation analysis of a region of transcription start region±1 kb.

FIG. 9 shows change in gene expression in cells treated with DMSO, an inhibitor (NCD38) or Im3-N.

FIG. 10 shows results of identifying histone acetylation after Im0-N or Im3-N treatment by ChIP sequencing. The abscissa of each graph shows results of counting the number of a WWWW sequence (Im0-N treatment) or the number of a WCGW sequence (Im3-N treatment) in a histone acetylation region. The ordinate of each graph shows the percent appearance of each count.

FIG. 11 shows results obtained by further analyzing the results of FIG. 10. The pie graphs (left two) of FIG. 11 show that as a result of treating each portion with Im0-N or Im3-N after classification into 36 or more (rich) and 12 or less (poor) in terms of the count of the WWWW sequence based on treatment with an inhibitor NCD38 alone, the rich portion is surely activated by Im0-N and rarely activated by Im3-N. The pie graphs (right two) of FIG. 11 show that as a result of treating each portion with Im0-N or Im3-N after classification into 5 or more (rich) and 3 or less (poor) in terms of the count of the WCGW sequence, the rich portion is surely activated by Im3-N and rarely activated by Im3-0.

### DETAILED DESCRIPTION OF EMBODIMENTS

In the present specification, the term “living subject” means a living body of an animal or plant, and a cell or a population regardless of whether or not to be separated from the living subject. The animal or plant is, for example, an animal, which is, for example, a vertebrate, for example, a mammal, for example, a primate, or, for example, a human.

In the present specification, the term “subject” is a mammal, for example, a primate, or, for example, a human. In the present specification, the subject affected by a cancer is also referred to as a subject having a (the) cancer. In the present specification, when the subject is a human, the subject is also referred to as a patient.

In the present specification, the term “DNA methylation” means methylation for a DNA molecule. For example, methylation for a carbon atom at position 5 of the pyrimidine ring of cytosine is known as the DNA methylation. The DNA methylation occurs at a CpG dinucleotide site on a DNA molecule. The DNA methylation is known to reduce the expression of a gene in the vicinity and has been found to play an important role in the growth of almost all types of cancers. For example, it is known that hypermethylation of DNA occurs in a promoter region of a cancer suppressor gene and represses the transcription of the cancer suppressor gene, thereby causing a cancer. Examples of the repression of gene expression by the methylation of a gene promoter region include repression of CDKN2A gene expression in human large intestine cancer, and repression of MGMT gene expression in brain tumor.

In the present specification, the term “promoter” means a transcription regulation region of a gene. The promoter is known to reside in an upstream region from −200 to −1, particularly, a region from −100 to −1, of the gene, when the transcription start site is defined as +1. For example, in a eukaryote, the promoter contains a TATA sequence called TATA box upstream from a −25 region. Also, the promoter contains a CAAT box in a region from −100 to −60 and a GC box in a region from −60 to −40. These sequences are well known to be related to gene transcription regulation. In the present specification, the position of DNA in the promoter region is indicated by a numeric as described above. The location of particular DNA at a relative position to the transcription start site is defined by a numeric in such a way that the location is represented by the numeric −1 as the position shifts upstream by one base from the transcription start point (site) defined as +1, and by the numeric 1 as the position shifts downstream by one base therefrom.

In the present specification, the term “gene expression” means the transcription of DNA to RNA. Examples of the RNA include messenger RNA (hereinafter, referred to as “mRNA”). Reduction in gene expression means that the transcription level to mRNA is reduced.

In the present specification, the term “pyrrole imidazole polyamide” is a linear molecule composed of a linkage of N-methylpyrrole units (hereinafter, abbreviated to “Py”) and N-methylimidazole units (hereinafter, abbreviated to “Im”), and refers to a polymer in which a linker (e.g., γ-aminobutyric acid) intervenes between the Py unit(s) and/or the Im unit(s) in the molecule, whereby the molecule takes a hairpin structure. In the present specification, the pyrrole imidazole polyamide is not in a form fused with a drug (including a promoter and an inhibitor) for histone protein modification, such as a histone deacetylation inhibitor, or for DNA modification, such as a DNA methylation inhibitor, unless otherwise specified.

The pyrrole imidazole polyamide is known to be capable of entering a minor groove of DNA where Im/Py binds to a G-C base pair, Py/Im binds to a C-G base pair, and Py/Py binds to an A-T base pair or a T-A base pair. Particularly, Py interacts strongly with G, whereby the pyrrole imidazole polyamide binds in a sequence-specific manner to the DNA minor groove. The Im unit may be changed to, for example, a β-alanine unit. The β-alanine unit can be contained at a ratio of 1 unit per 3 to 4 Py units or Im units.

Thus, those skilled in the art can readily design pyrrole imidazole polyamide binding in a sequence-specific manner to target DNA, on the basis of the sequence of the target DNA, because the relation described above has been technically established.

The term “Py/Im” means that Py and Im are positioned in proximity in the molecule having a hairpin structure by folding, even though they reside at distant positions in the arrangement of the Py units and the Im units in the molecule.

The pyrrole imidazole polyamide is represented by the general formula (A):

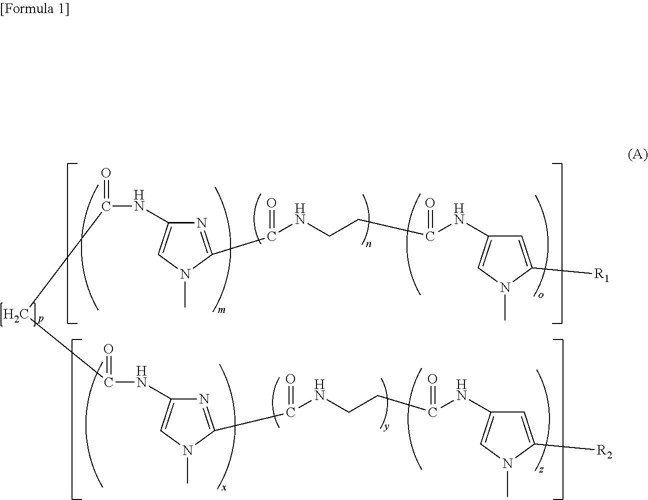

wherein m, n, o, p, x, y, and z are each independently a natural number, and R₁ and R₂ are each independently a protective group.

The general formula (A) is simplified as R₂—[(Py)_{z}-β_{y}-(Im)ₓ]-(CH₂)ₚ-[(Im)ₘ-βₙ-(Py)ₒ]-R₁ wherein m, n, o, p, x, y, and z are each independently a natural number, and R₁ and R₂ are each independently a protective group, according to convention.

In the general formula (A), each unit is described such that the same type of unit is included in one block as shown therein, for the sake of convenience. In actuality, the arrangement of the Py units, the Im units, and the β-alanine units is changed so as to correspond to the sequence of the DNA as the binding target according to the rule mentioned above. In the general formula (A), the R₁ group can be, for example, —CO—NH—CH₂—CH₂—CO—NH—CH₂—CH₂—CH₂—N(CH)₂. The R₂ group can be, for example, —NH—CO—CH. In the general formula (A), p can be a natural number of 2 to 4, for example, 3, and m+n+o and x+y+z can be 5 to 20, 7 to 15, or 7 to 10 according to the length of the DNA as the binding target.

The compound of the formula (I) is exemplary pyrrole imidazole polyamide and is a molecule composed of a linkage of the Py units and the Im units via —CO—NH—. A linker γ-aminobutyric acid intervenes between the Py and Im units in the molecule so that the molecule is folded at the linker moiety, whereby the molecule takes a hairpin structure as a whole. The pyrrole imidazole polyamide takes a hairpin structure in a minor groove of DNA and stabilizes the binding through the interaction between the bases of the DNA and the Py units or the Im units. This compound of the formula (I) can bind in a sequence-specific manner to a 7-base long region from −19 to −13 counted from the transcription start site of MLH1 gene.

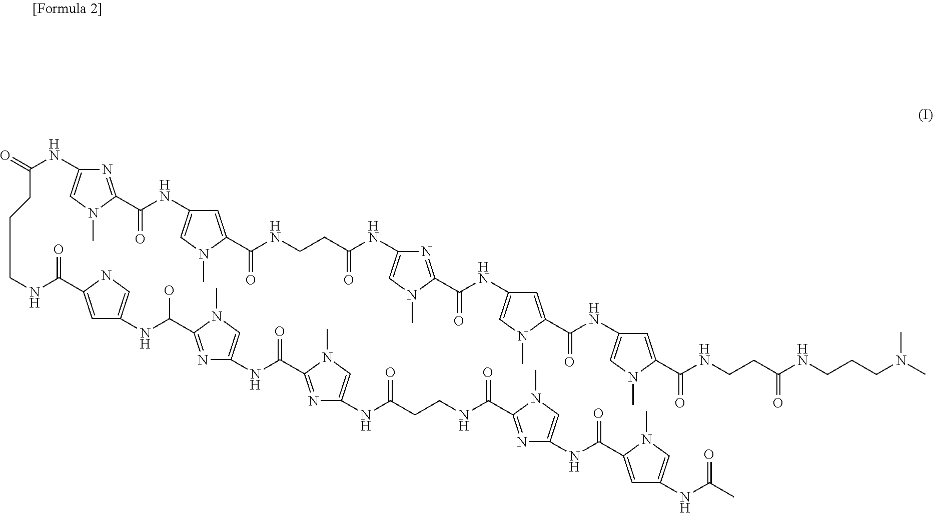 [SMILES: CC(=O)Nc1cc(C(=O)Nc2cn(C)c(C(=O)NCCC(=O)Nc3cn(C)c(C(=O)Nc4cn(C)c(C(O)Nc5c[nH]c(C(=O)NCCCC(=O)Nc6cn(C)c(C(=O)Nc7cc(C(=O)NCCC(=O)Nc8cn(C)c(C(=O)Nc9cc(C(=O)Nc%10cc(C(=O)NCCC(=O)NCCCN(C)C)n(C)c%10)n(C)c9)n8)n(C)c7)n6)c5)n4)n3)n2)n(C)c1]

The compound of the formula (I) is also abbreviated as given below according to convention, because the orders of the Py units and the Im units in the sequence has technical significance for sequence-specific binding to DNA, as shown below. In the abbreviation given below, the sequence is described on a straight line. The binding to DNA is performed by forming a hairpin structure at the “γ” moiety. In the present specification, this sequence is also referred to as a primary structure.

Ac-Py-Im-β-Im-Im-Py-γ-Im-Py-β-Im-Py-Py-β-Dp [Formula 3]

wherein Ac represents an acetyl group, β represents β-alanine, γ represents γ-aminobutyric acid, and Dp represents diaminopropane.

In the present specification, the term “sequence-specific” is used to mean binding to the target sequence as designed, or stronger binding to the target sequence than that to other sequences, because pyrrole imidazole polyamide binds in a sequence-dependent manner to DNA. For enhancing the sequence specificity of the interaction between pyrrole imidazole polyamide and DNA, the target DNA sequence is preferably longer and can be, for example, 4 bases or longer, 5 base pairs or longer, 6 base pairs or longer, 7 base pairs or longer, 8 base pairs or longer, 9 base pairs or longer, 10 base pairs or longer, or further.

In the present specification, the term “demethylation treatment” means a treatment to remove the methylation of a carbon atom at position 5 of the pyrimidine ring of cytosine.

In the present specification, the term “inhibition treatment of deacetylation” means a treatment to inhibit the deacetylation of histone. The deacetylation treatment is performed by, for example, the inhibition of histone deacetylase (HDAC).

In the present specification, the term “treatment” is used to mean that both therapy and prevention are included therein.

The present inventors have found that: pyrrole imidazole polyamide whose target sequence is only 4 base pairs (e.g., 7 base pairs) on a promoter region suppresses DNA methylation in the wide neighborhood of the target site on the promoter; and this cancels transcriptional repression of the gene that is driven by the promoter. No special DNA methylation inhibitor is necessary therefor. This is presumably because the pyrrole imidazole polyamide binds to the promoter region in a living subject and thereby widely changes DNA conformation, widely influencing DNA methylation reaction or transcription reaction.

Accordingly, the present invention provides a composition for use in inhibiting repression of gene expression by DNA methylation in a living subject (or a gene transcriptional repression inhibitor), comprising pyrrole imidazole polyamide, wherein the pyrrole imidazole polyamide is designed so as to bind in a sequence-specific manner to a minor groove of promoter region DNA of the gene. In a certain aspect, the composition contains no DNA methylation inhibitor other than the pyrrole imidazole polyamide.

Pyrrole imidazole polyamide having a linkage of N-methylpyrrole units (hereinafter, referred to as Py) and N-methylimidazole units (hereinafter, referred to as Im) via amide bonds and containing one linker 1 and one or more linkers 2 between the Py unit(s) and/or Im unit(s), the pyrrole imidazole polyamide taking a hairpin structure by folding at the site of the linker 1 within a minor groove of DNA where Im/Py binds to a G-C base pair of the DNA, Py/Im binds to a C-G base pair, and Py/Py binds to an A-T base pair or a T-A base pair, wherein Py may be replaced with β-alanine, and the pyrrole imidazole polyamide wherein the linker 1 is γ-aminobutyric acid can be used. The replacement of Py with β-alanine can be performed at a ratio of one per 3 to 4 constituent units. If the target DNA sequence to be bound is determined, pyrrole imidazole polyamide binding in a sequence-specific manner to the target DNA can be designed, as mentioned above. As is evident from the chemical structure of the pyrrole imidazole polyamide, it is known that this molecule may be synthesized on a solid phase by a Boc method or a Fmoc method well known to those skilled in the art and can be synthesized using a commercially available automatic synthesizer or the like.

The present inventors have found that pyrrole imidazole polyamide preferably inhibits DNA methylation in an environment where the methylation is promoted. Although cells cultured, for example, under hypoxic conditions, after DNA demethylation treatment or after DNA demethylation treatment and histone demethylation treatment increase DNA methylation by the action of their own DNA methyltransferase (see, for example, Lu, Y. et al., Cell Reports, 8: 501-513, 2014), the present inventors have also found that pyrrole imidazole polyamide inhibits the DNA methylation in this respect. Accordingly, the composition of the present invention may be used after DNA demethylation treatment or after DNA demethylation treatment and histone demethylation treatment. The composition of the present invention can also be used, for example, in inhibiting DNA methylation that is induced under hypoxic conditions and can be used, for example, in inhibiting DNA methylation that is induced under hypoxic conditions after DNA demethylation treatment or after DNA demethylation treatment and histone demethylation treatment. Chronic inflammation caused by Helicobacter pylori infection or the like is known as an environment, other than hypoxia, where DNA methylation is promoted within a living subject (see, for example, Matsusaka, K. et al., World J. Gastroenterol., 20 (14): 3916-3926, 2014). Also, infection by some pathogens such as Epstein-Barr virus infection promotes DNA methylation (see, for example, Matsusaka, K. et al., 2014, supra). DNA methylation may also be promoted by abnormalities in cells themselves of a living subject, such as a rise in the expression of DNA methyltransferase (DNNT), IDH gene abnormality, and reduction in the activity of TET2 enzyme (see, for example, Figueroa, M E. et al., Cancer Cell, 18: 553-567, 2010; and Turcan S., Nature, 483: 479-483).

The present inventors have also found that pyrrole imidazole polyamide, particularly, after DNA demethylation treatment and histone demethylation treatment, at least partially cancels transcriptional repression of a gene by subsequent DNA methylation in the promoter region and can maintain transcription. Accordingly, the composition of the present invention may be used after DNA demethylation treatment and inhibition treatment of histone deacetylation.

The DNA methylation treatment can be performed using, for example, a DNA methylation inhibitor. Examples of the DNA methylation inhibitor include DNA methyltransferase inhibitors. In the present invention, the DNA methylation inhibitor is not particularly limited as long as the DNA methylation inhibitor can inhibit DNA methylation. Examples thereof include anticancer agents such as 5-aza-2′-deoxycytidine and 5-azacytidine, any of which can be used in the present invention.

The inhibition treatment of histone deacetylation can be performed using, for example, a HDAC inhibitor. Examples of the HDAC inhibitor include, but are not particularly limited to, trichostatin A, n-butyric acid, apicidin vorinostat (SAHA), and valproic acid, any of which can be used in the present invention.

In the present invention, the gene whose repression of expression by DNA methylation is to be inhibited (target gene) is, for example, a gene having methylated promoter region DNA. Examples of the gene having methylated promoter region DNA include cancer suppressor genes having methylated promoter region DNA. Such genes are well known to those skilled in the art. Examples of the gene or the cancer suppressor gene having methylated promoter region DNA include MLH gene, BRCA1 gene, estrogen receptor gene, CDKN2A gene, MGMT gene, CDH1 gene, RUNX3 gene, RASSF1A gene, and SFRP1 gene.

The pyrrole imidazole polyamide can be designed so as to bind in a sequence-specific manner to a part of DNA in a promoter region (e.g., a ±1 kb region or a region from −200 to −1, preferably a region from −100 to −1) of any of these target genes. The pyrrole imidazole polyamide can be designed so as to bind in a sequence-specific manner to, for example, a portion (a region of 5 or more bases long as mentioned above) of a region from −200 to −1, preferably a region from −100 to −1, more preferably a region from −50 to −1, of the MLH1 gene.

Pyrrole imidazole polyamide that can bind in a sequence-specific manner to the promoter region of the MLH1 gene, for example, a compound represented by the formula (I), can be used as the pyrrole imidazole polyamide that inhibits methylation of the promoter region of the MLH1 gene. A conjugate of the pyrrole imidazole polyamide described above and a histone modifying enzyme inhibitor can be used as a conjugate of the pyrrole imidazole polyamide that inhibits methylation of the promoter region of the MLH1 gene, and a histone modifying enzyme inhibitor.

In a certain aspect, the present invention provides a conjugate of pyrrole imidazole polyamide and a histone modifying enzyme inhibitor. The length of the target DNA sequence of the pyrrole imidazole polyamide in the conjugate can be, for example, 4 bases or longer, 5 base pairs or longer, 6 base pairs or longer, 7 base pairs or longer, 8 base pairs or longer, 9 base pairs or longer, 10 base pairs or longer, or further, as mentioned above. The length of the target DNA sequence of the pyrrole imidazole polyamide in the conjugate can be 7 base pairs or shorter, 6 base pairs or shorter, 5 base pairs or shorter, or 4 base pairs. In a certain aspect of the present invention, the histone modifying enzyme inhibitor can be an inhibitor against histone methylase, a histone acetyltransferase activator (HAT activator) or a histone deacetylase inhibitor (HDAC inhibitor). In a certain aspect of the present invention, the histone modifying enzyme inhibitor is an inhibitor against a lysine-specific demethylase LSD1. Examples of the inhibitor against LSD1 include NCD38 and GSK-LSD1, any of which can be used in the present invention. In a certain aspect of the present invention, examples of the HDAC inhibitor include trichostatin A (TSA), butyric acid, apicidin, valproic acid, LBH589 and suberoylanilide hydroxamic acid (SAHA). In a certain aspect of the present invention, examples of the HAT activator include N-(4-chloro-3-trifluoromethylphenyl)-2-ethoxy-6-pentadecylbenzamide (CTBP) and derivatives thereof, for example, N-(4-chloro-3-trifluoromethylphenyl)-2-ethoxy-benzamide (CTB).

In a certain aspect, the present invention provides a composition for reducing DNA methylation, comprising a conjugate of pyrrole imidazole polyamide and a histone modifying enzyme inhibitor. In a certain aspect, the present invention provides a composition for use in improving an expression level of a target gene, comprising a conjugate of pyrrole imidazole polyamide and a histone modifying enzyme inhibitor, wherein the pyrrole imidazole polyamide is designed so as to bind in a sequence-specific manner to partial DNA of a promoter region (e.g., a ±1 kb region or a region from −200 to −1, preferably a region from −100 to −1) of the target gene.

In another aspect, the present invention provides a pharmaceutical composition for treating a cancer in a subject with the cancer. Since a cancer suppressor gene having methylated promoter region DNA is transcriptionally repressed, the amount of a gene product produced from the cancer suppressor gene is decreased, causing a cancer. Thus, the pharmaceutical composition for treating a cancer in a cancer patient according to the present invention, comprises pyrrole imidazole polyamide binding in a sequence-specific manner to a promoter region of a cancer suppressor gene having methylated promoter region DNA, or a conjugate of the pyrrole imidazole polyamide and a histone modifying enzyme inhibitor. The pharmaceutical composition of the present invention inhibits the methylation of the promoter region DNA of the cancer suppressor gene and can thereby suppress transcriptional repression of the cancer suppressor gene so that the cancer can be treated.

Methylated promoter region DNA of a cancer suppressor gene is known in almost all cancers and causes the cancers. For example, as for the MLH1 gene, methylated promoter region DNA of the cancer suppressor gene is partly responsible for large intestine cancer (colorectal cancer), stomach cancer, esophageal cancer, head and neck squamous cell carcinoma, lung cancer, pancreatic cancer, liver cancer, bile duct cancer, blood tumor, breast cancer, uterine corpus cancer, ovary cancer, brain tumor, and soft tissue sarcoma. Accordingly, the pharmaceutical composition of the present invention can be used in treating large intestine cancer (colorectal cancer), stomach cancer, esophageal cancer, head and neck squamous cell carcinoma, lung cancer, pancreatic cancer, liver cancer, bile duct cancer, blood tumor, breast cancer, uterine corpus cancer, ovary cancer, brain tumor, and soft tissue sarcoma. Also, methylated promoter region DNA of the MLH1 gene is known to cause these cancers. Thus, in the pharmaceutical composition of the present invention, the target gene of the pyrrole imidazole polyamide can be MLH1 gene.

In cancer patients, it is known that DNA methylation is increased in the promoter region of the cancer suppressor gene, and the degree of DNA methylation is elevated along with the onset or progression of the cancer. According to the present inventors, the pyrrole imidazole polyamide or the conjugate of pyrrole imidazole polyamide and a histone modifying enzyme inhibitor was able to particularly suppress the progression of DNA methylation after DNA demethylation treatment or after DNA demethylation treatment and inhibition treatment of histone deacetylation. Accordingly, the pharmaceutical composition of the present invention can be used in combination with a DNA demethylating agent or a combination of a DNA demethylating agent and a histone deacetylation inhibitor (hereinafter, also referred to as a “concomitant agent”). Alternatively, the pharmaceutical composition of the present invention may be administered to a subject that has undergone therapy with a DNA demethylating agent or a combination of a DNA demethylating agent and a histone deacetylation inhibitor, as a subject to be treated.

Examples of the DNA demethylating agent that can be used in combination with the pharmaceutical composition of the present invention include, but are not particularly limited to, anticancer agents such as azacytidine and decitabine. Examples of the histone deacetylation inhibitor that can be used in combination with the pharmaceutical composition of the present invention include trichostatin A, n-butyric acid, apicidin and valproic acid.

The order of administration of the pharmaceutical composition of the present invention and the DNA demethylating agent or the combination of the DNA demethylating agent and the histone deacetylation inhibitor is not particularly limited. For example, the DNA demethylating agent or the combination of the DNA demethylating agent and the histone deacetylation inhibitor can be administered, and then, the pharmaceutical composition of the present invention can be administered.

The pharmaceutical composition of the present invention can be administered to a patient who has been subjected to anticancer agent therapy with a DNA demethylating agent or a DNA demethylating agent and a histone deacetylation inhibitor. Since the pharmaceutical composition of the present invention can suppress DNA methylation, this approach is capable of reducing the dose or dosing interval of the DNA demethylating agent or the DNA demethylating agent and the histone deacetylation inhibitor and can mitigate biotoxicity known to be caused by these drugs. Accordingly, for example, the pharmaceutical composition of the present invention can be administered according to a dosing regime in which a plurality of cycles are performed, each involving administering the concomitant agent to a cancer patient and then administering the pharmaceutical composition of the present invention.

According to the present invention, use of pyrrole imidazole polyamide binding in a sequence-specific manner to promoter region DNA of the MLH1 gene, or a conjugate of the pyrrole imidazole polyamide and a histone modifying enzyme inhibitor can cancel the transcriptional repression of the MLH1 gene, or at least partially maintain the gene expression of the MLH1 gene. Accordingly, the present invention provides a pharmaceutical composition for use in preventing the onset of a cancer in a subject with increased methylation of MLH1 gene, or for use in reducing a risk of developing a cancer in the subject, comprising pyrrole imidazole polyamide binding in a sequence-specific manner to promoter region DNA of MLH1 gene, or a conjugate of the pyrrole imidazole polyamide and a histone modifying enzyme inhibitor. This pharmaceutical composition can also be used in combination with the concomitant agent described above.

Examples of the subject with increased methylation of MLH1 gene include colorectal serrated adenocarcinoma. The subject with colorectal serrated adenocarcinoma, when left untreated, develops large intestine cancer due to transcriptional repression of the MLH1 gene resulting from the increased methylation of the MLH1 gene. Thus, the pharmaceutical composition of the present invention can be administered to such a subject to thereby prevent the onset of large intestine cancer or reduce a risk of developing large intestine cancer.

The present invention provides a method for inhibiting repression of gene expression by DNA methylation in a living subject, comprising

contacting pyrrole imidazole polyamide or a conjugate of pyrrole imidazole polyamide and a histone modifying enzyme inhibitor with the living subject, or administering pyrrole imidazole polyamide or a conjugate of pyrrole imidazole polyamide and a histone modifying enzyme inhibitor to the living subject, wherein

the pyrrole imidazole polyamide or the conjugate of pyrrole imidazole polyamide and a histone modifying enzyme inhibitor is designed so as to bind in a sequence-specific manner to a minor groove of promoter region DNA of the gene. This method may further comprise contacting a DNA demethylating agent with the living subject, or administering a DNA demethylating agent to the living subject, or may further comprise contacting a DNA demethylating agent with the living subject, or administering a DNA demethylating agent to the living subject, and contacting a HDAC inhibitor with the living subject, or administering a HDAC inhibitor to the living subject.

The method of the present invention provides a method for treating a cancer in a subject with the cancer, comprising

administering the pharmaceutical composition of the present invention to the subject.

The present invention provides a method for preventing the onset of a cancer in a subject with increased methylation of MLH1 gene, or reducing a risk of developing a cancer in the subject, comprising administering the pharmaceutical composition of the present invention to the subject.

The present invention provides use of pyrrole imidazole polyamide or a conjugate of pyrrole imidazole polyamide and a histone modifying enzyme inhibitor for manufacturing the pharmaceutical composition of the present invention, wherein the pyrrole imidazole polyamide is designed so as to bind to a promoter region of a cancer suppressor gene.

The composition or the pharmaceutical composition of the present invention may comprise pyrrole imidazole polyamide binding in a sequence-specific manner to one site in the promoter region of the target gene, or a conjugate of the pyrrole imidazole polyamide and a histone modifying enzyme inhibitor, or may comprise a plurality of different pyrrole imidazole polyamides respectively binding in a sequence-specific manner to a plurality of different sites, or a conjugate of a pyrrole imidazole polyamide and a histone modifying enzyme inhibitor. The composition or the pharmaceutical composition of the present invention may comprise a conjugate comprising pyrrole imidazole polyamide linked to a DNA methylation inhibitor or a histone modifying enzyme inhibitor such as a HDAC inhibitor via a linker.

The pharmaceutical composition of the present invention may contain an excipient. The pharmaceutical composition of the present invention can be administered by, for example, local administration (e.g., intratumoral administration), intravenous administration, and percutaneous administration. Those skilled in the art can appropriately select an excipient and can appropriately formulate the pharmaceutical composition of the present invention.

Hereinafter, the invention will be described with reference to Examples. However, the present invention is not limited by the specific contents given below. The present invention is an invention specified by the scope of claims.

### EXAMPLES

### Example 1: Design and Preparation of Pyrrole Imidazole Polyamide Against Promoter Region of MLH1 Gene

The MLH1 gene is a cancer suppressor gene in which a CpG island of the gene promoter region is known to undergo methylation, whereby the transcription of the MLH1 gene is inactivated. In this Example, pyrrole imidazole polyamide against the promoter region of the MLH1 gene was designed and prepared.

The pyrrole imidazole polyamide is a polymer compound obtained by polymerizing N-methylpyrrole units (referred to as Py) and N-methylimidazole units (referred to as Im). Usually, the pyrrole imidazole polyamide takes a U-shaped conformation by folding at the site of a γ-aminobutyric acid unit (referred to as γ) and binds to a minor groove of DNA. Im strongly binds to a G base, and Py binds to C, A and T bases. Therefore, pyrrole imidazole polyamide specifically binding to a sequence of target DNA can be designed by properly arranging Py and Im according to the DNA sequence. Py may be replaced with β-alanine. Appropriate introduction of β-alanine (e.g., every 3 or 4 base pairs) allows the pitch of the DNA to match the pitch of the pyrrole imidazole polyamide.

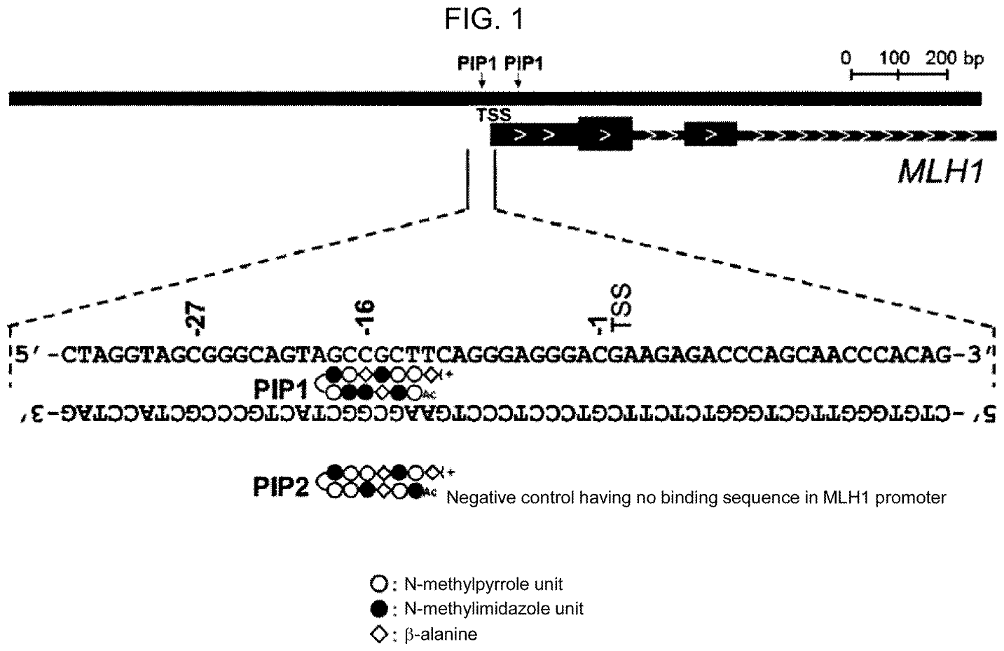

Here, pyrrole imidazole polyamide specific for 7 bases from −19 to −13 of the MLH1 gene was synthesized (see FIG. 1 and the compound of the formula (I)) (hereinafter, this pyrrole imidazole polyamide is referred to as PIP1). Here, these numerics indicate positions on the gene in such a way that each position is represented by the numeric −1 as the position shifts upstream by one base from the transcription start site defined as +1 (the same holds true for the description below).

The pyrrole imidazole polyamide may be readily synthesized by a solid phase synthesis method such as a Hoc method or a Fmoc method and can also be synthesized using a commercially available automatic synthesizer or the like. In this Example, PIP1 was obtained by synthesis by Hipep Laboratories (Kyoto, Japan) with reference to the designed pyrrole imidazole polyamide. PIP2 having no binding site on the MLH1 gene (see FIG. 1 and a compound of the formula (II)) was used as a negative control. The pyrrole imidazole polyamide was stored at a concentration of 20 mM in dimethyl sulfoxide (DMSO).

[Formula 4]

PIP2: Ac-Im-Py-β-Im-Py-Py-γ-Im-Py-Py-β-Im-Py-β-Dp (II)

wherein the symbols are as defined above.

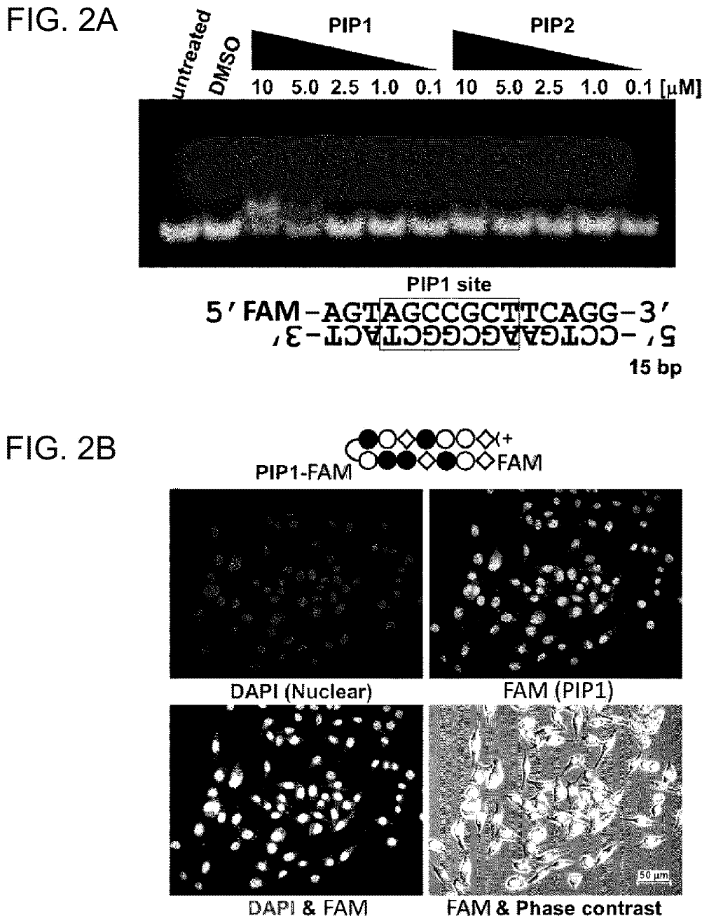

PIP1 was confirmed by gel shift assay to bind to the target sequence. FAM-labeled double-stranded oligo DNA of 15 base pairs including a region from −22 to −8 of the MLH1 gene (double-stranded oligo DNA obtained by annealing sequences of SEQ ID NOs: 2 and 3) was prepared (lower part of FIG. 2A). 1 μM double-stranded oligo DNA was mixed at an indicated final concentration with PIP1 or PIP2 dissolved in 10 μL of a reaction solution containing 10 mM Tris-HCl (pH 8.0) and 10% DMSO, followed by incubation at 37° C. for 1 hour. The obtained reaction product was electrophoresed using 20% polyacrylamide gel, and double-stranded DNA on the gel was visualized using LAS-3000 (Fujifilm Corp., Japan).

The results were as shown in FIG. 2A. The band of the double-stranded DNA shifted upward only when PIP1 was added. PIP1 was thereby able to be confirmed to bind to the target double-stranded DNA.

### Example 2: Intracellular Localization of PIP1

In this Example, the intracellular localization of PIP1 was confirmed. In this Example, the intracellular localization within a large intestine cancer cell line RKO was confirmed.

1.0×10⁵ cells of the RKO cells were seeded over a 35 mm dish and cultured in 2 mL of a medium (Eagle's MEM medium supplemented with 10% fetal bovine serum, 100 units/mL of penicillin, and 100 μg/mL of streptomycin). 24 hours later, the cells were mixed with FAM-labeled 1 μM PIP1 (FAM-β-Py-Im-β-Im-Im-Py-γ-Im-Py-β-Im-Py-Py-β-Dp), incubated for 24 hours, and fixed for 2 hours on ice in 10% formaldehyde. The fixed cells were washed with PBS, nuclear-stained with 1 μg/mL of 4′,6-diamidino-2-phenylindole (DAPI), and then observed under a fluorescence microscope BZ-X710 (KEYENCE Corp., Osaka, Japan).

The results were as shown in FIG. 2B. As shown in FIG. 2B, the localization of PIP1 was completely consistent with the nucleus. This demonstrated that PIP1 enters a cell through the cell membrane from a medium, and furthermore, is enriched and distributed in the nucleus.

### Example 3: PIP1 and DNA Methylation

In this Example, PIP1 was studied for its influence on methylation of the MLH1 gene.

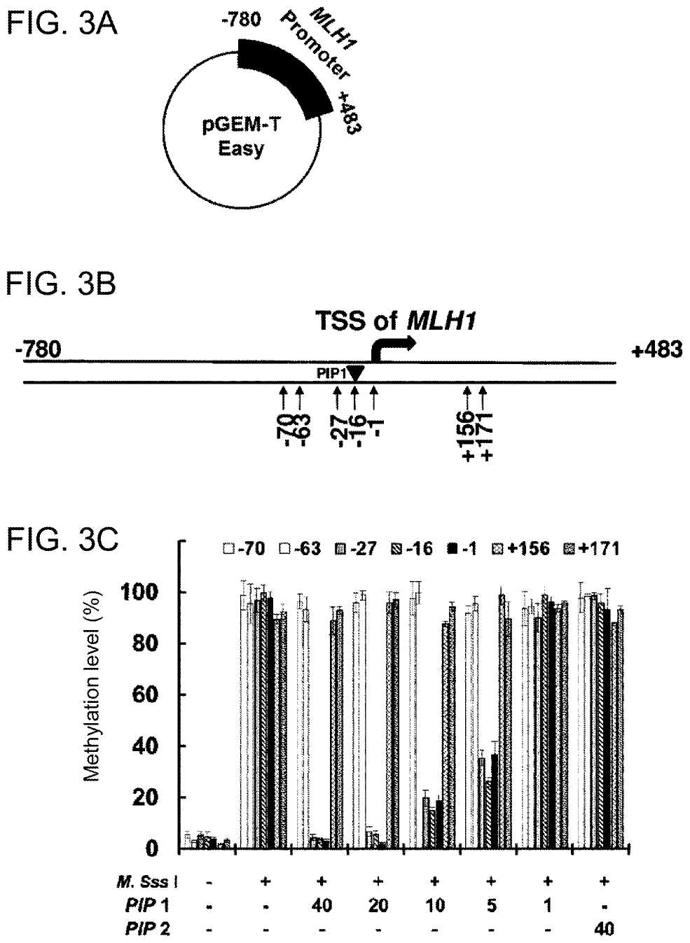

A region from −780 to +483 (SEQ ID NO: 1) of MLH1 was amplified from the genomic DNA of the RKO cells by PCR using primers of SEQ ID NOs: 6 and 7 to obtain a DNA fragment, which was then integrated into pGEM-T Easy vector (Promega Corp., Fitchburg, USA) according to the manufacturer's manual (see FIG. 3A). 1 μg of the obtained plasmid was incubated at 25° C. for 12 hours in varying concentrations of PIP1 or 40 μM of PIP2 and 1× NEB buffer solution 2 (New England Biolabs Inc., Ipswich, USA) to bind the pyrrole imidazole polyamide to the DNA. For a negative control, DMSO was added to the plasmid and incubated.

320 μg of S-adenosylmethionine (New England Biolabs Inc.) and 4 units of CpG methyltransferase M.SssI (New England Biolabs Inc.) were added to each sample, and 5% DMSO was added thereto to adjust the amount to 20 μL. After incubation at 37° C. for 2 hours, the reaction was terminated by incubation at 65° C. for 20 minutes. The DNA was purified by ethanol precipitation and dissolved in 100 μL of 10 mM Tris-HCl (pH 8.0). Then, the degree of DNA methylation at various sites of the MLH1 gene was tested by pyrosequencing. The primers used were as described in the following table.

TABLE 1

| Primer name and sequence | SEQ ID NO |
|---|---|
| Forward primer for amplification of region from −27 to −1: | SEQ ID NO: 8 |
| 5′-GGTATTTTTGTTTTTTATTGGTTGGATATT-3′ | |
| Reverse primer for amplification of region from −27 to −1: | SEQ ID NO: 9 |
| 5′-^{B}AATACCAATCAAATTTCTCAACTCTAT-3′ | |
| Primer for sequencing of region from −27 to +1: | SEQ ID NO: 10 |
| 5′-GTAAGAGTATATTTTTTTAGGT-3′ | |
| Forward primer for amplification of region from −70 to −63: | SEQ ID NO: 11 |
| 5′-GGTATTTTTGTTTTTATTGGTTGGATATT-3′ | |
| Reverse primer for amplification of region from −70 to −63: | SEQ ID NO: 12 |
| 5′-^{B}AATACCAATCAAATTTCTCAACTCTAT-3′ | |
| Primer for sequencing of region from −70 to −63: | SEQ ID NO: 13 |
| 5′-TTTTAAAAAAGAATTAATAGGAAGA-3′ | |
| Forward primer for amplification of region from +157 to +172: | SEQ ID NO: 14 |
| 5′-AGGTGATTGGTTGAAGGTATTT-3′ | |
| Reverse primer for amplification of region from +157 to +172: | SEQ ID NO: 15 |
| 5′-^{B}CCAATTCTCAATCATCTCTTTAATAACAT-3′ | |
| Primer for sequencing of region from +157 to +172: | SEQ ID NO: 16 |
| 5′-GATTGGTTGAAGGTATTTT-3′ | |

*In the table, “B” denotes biotinylation.

The results were as shown in FIG. 3C. As shown in FIG. 3D, PIP1 inhibited DNA methylation in a concentration-dependent manner in the region from −27 to −1 of the MLH1 gene.

### Example 4: Influence of PIP1 on Methylation of Promoter Region of MLH1 Gene in RKO Cell

In this Example, PIP1 was studied for its methylation suppressive effect using cells.

1.0×10⁵ cells of the RSO cells were seeded over a 6 cm dish and treated with a medium containing 5.0 μM 5-aza-2′-deoxycytidine (AZA) over 7 days from the next day. The medium was replaced with a fresh medium every 24 hours. The cells were treated with a medium containing 10 nM trichostatin A (TSA) on the 7th day and recovered on the 8th day.

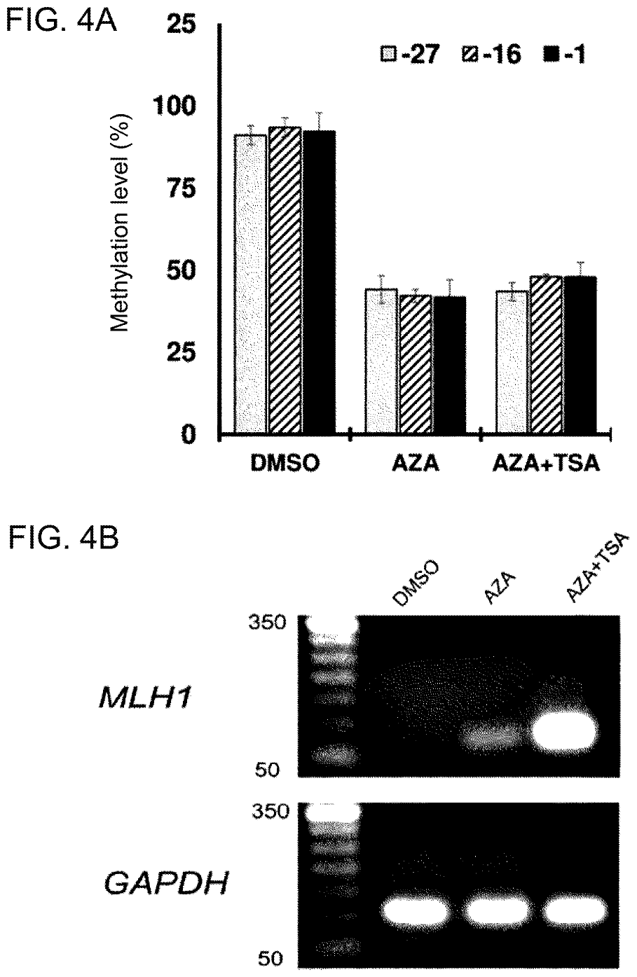

The degree of methylation in the promoter region from −27 to −1 of the MLH1 gene was confirmed by pyrosequencing in the same way as in Example 3. The results were as shown in FIG. 4A. As shown in FIG. 4, the degree of methylation was reduced in this region both by the treatment with AZA alone and by the treatment with TSA in addition to AZA. As a result of then confirming the expression of mRNA of the MLH1 gene by RT-PCR (primer sequences: SEQ ID NOs: 2 and 3), the expression was restored both by the AZA treatment and by the AZA and TSA treatment (see FIG. 4B). For a control, the expression of GAPDH was confirmed by RT-PCR (primer sequences: SEQ ID NOs: 4 and 5).

The cells of the 8th day were cultured under hypoxic conditions of 1% O₂ and 5% CO₂. The cells were treated with 0.1% DMSO containing 5 μM PIP1 or PIP2, recovered on the 0th day (R0), the 20th day (R20) and the 30th day (R30), and examined for the degree of methylation in the region from −27 to −1 of the MLH1 gene by pyrosequencing. During the treatment with PIP, the medium containing PIP1 or PIP2 was replaced every 5 days, and the cells were subcultured when becoming confluent.

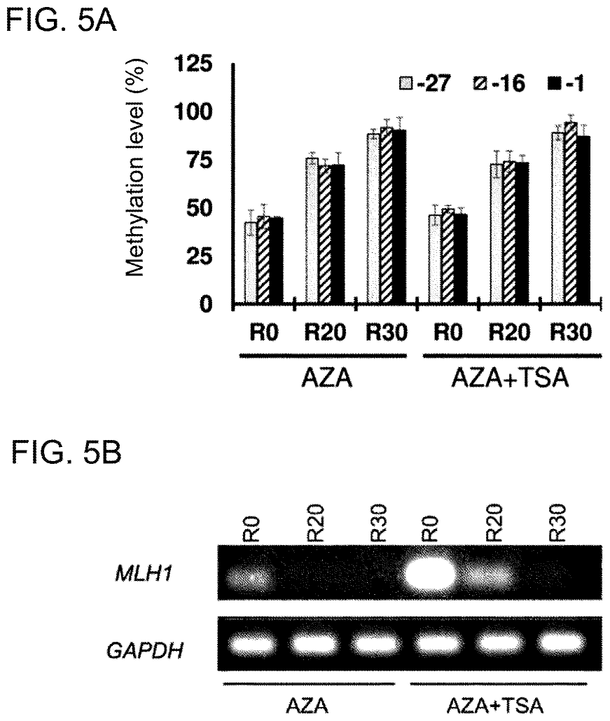

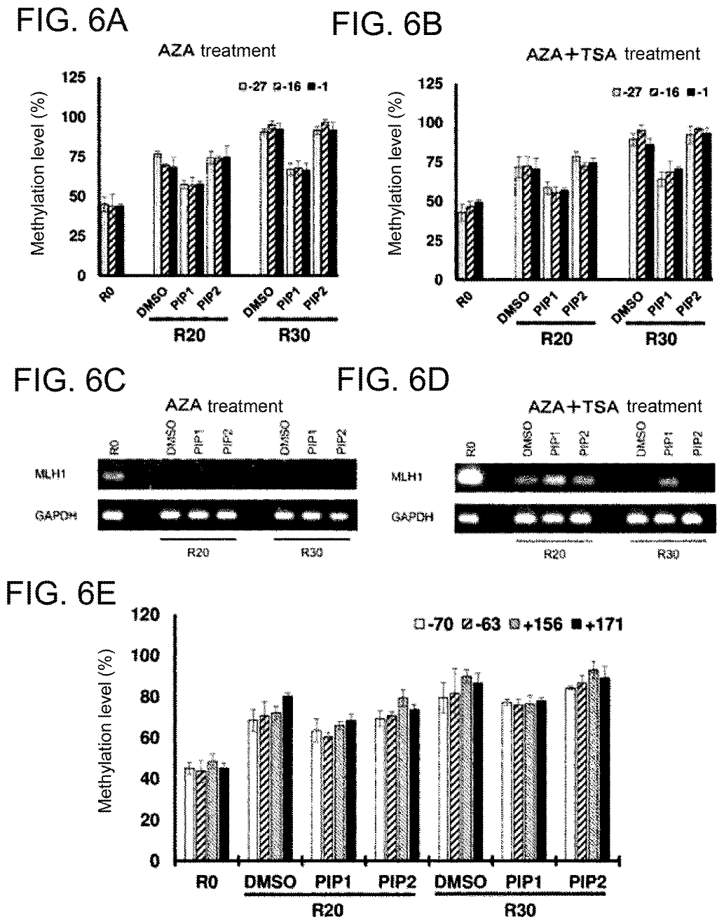

The results were as shown in FIGS. 5 and 6. As shown in FIG. 5, it is evident that without the PIP treatment, the methylation level was restored with time, and the MLH1 gene expression was also reduced with time. As shown in FIGS. 6A and 6B, the degree of DNA methylation was increased as a whole on R20 and R30 in the PIP-untreated group, whereas an increase in the degree of methylation was suppressed in the PIP1-treated cells. As shown in FIGS. 6C and 6D, in the AZA+TSA-treated cells, the expression of mRNA of the MLH1 gene, which is reduced along with methylation, was maintained by the PIP1 treatment. As a result of further confirming the degree of methylation in a region from −70 to +171 of the MLH1 gene, as shown in FIG. 6E, PIP1 was confirmed to suppress methylation in a −70 region, a −63 region, a +156 region, and a +171 region, which were sites considerably distant from the target site of PIP1.

These results demonstrated that PIP1 can be enriched and distributed in the nucleus in a cell, can suppress methylation of a target site in a cell, and, surprisingly, is capable of suppressing methylation of a DNA region in the neighborhood of the target site. These results also demonstrated that PIP1 can thereby cancel transcriptional repression of the gene by methylation. PIP can presumably be used in cancer management by canceling transcriptional repression of a cancer suppressor gene.

Since AZA or TSA is highly biotoxic, the dependence of treatment on these drugs cannot be elevated. According to the present invention, the effect of AZA or ISA treatment can be sustained by the administration of PIP after the AZA and/or TSA treatment. This is considered to provide a therapy with reduced burdens on living subjects.

### Example 5B: Inhibition of Methylation by Four-Base Recognition-Type PIP

In this Example, the methylation inhibitory effect of four-base recognition-type PIP was confirmed.

As shown in FIG. 7, motifs of W (i.e., A or T) recognition, C recognition, or G recognition were combined to synthesize PIP. Specifically, Me-Py-Py-Py-Py-β-Dp was synthesized as Im0, and Me-Py-Py-Im-Py-β-Dp was synthesized as Im3 (see J. Am. Chem. Soc. 1996, 118, 6141) {wherein β represents β-alanine, Me represents a methyl group, and Dp represents dimethylaminopropylamide}. Im0 forms a homodimer on a DNA sequence and recognizes an A/T sequence (i.e., 5′-WWWW-3′) without recognizing a CpG site. Im3 forms a homodimer on a DNA sequence and recognizes the CpG site (i.e., 5′-WCGW-3′).

The large intestine cancer cell line RKO cells were seeded at a density of 1×10⁵ cells over a 6 cm dish and incubated for 24 hours. Subsequently, the culture solution was replaced with a culture solution containing 0.5 μM 5-aza-2′-deoxycytidine (AZA). The cultured cells were exposed to AZA over 2.4 hours. The cells were recovered on the 10th day, or cultured under hypoxic conditions as mentioned below.

In order to culture the cells under hypoxic conditions, the cells were cultured for 8 days under continuous aeration conditions with humidified air containing 94% N₂, 5% CO₂ and 1% O₂ in an incubator equipped with an O₂ sensor and MCO-18M regulator (Sanyo Electric Co., Ltd., Osaka, Japan). The CO₂ concentration was maintained by the internal CO₂ concentration regulation mechanism. Subsequently, the AZA-treated cells were treated with 0.1% DMSO containing 10 μM Im0 or Im3 for 75 days. During this operation, the medium containing PIP was replaced every 5 days. The cells were subcultured at a density of 2×10⁵ cells over a 6 cm dish when becoming confluent. The cells were recovered on the 0th day (H0, before hypoxia treatment) and the 75th day (H75).

Genome-wide DNA methylation analysis was conducted as follows: first, 500 ng of genomic DNA obtained from each sample was treated with bisulfite using Zymo EZ DNA Methylation Kit (Zymo Research Corp., Irvine, Calif., USA). Whole genome amplification, labeling, hybridization and scanning were performed according to manufacturers' manuals.

Methylation analysis was conducted using Infinium Human Methylation 450 BeadChip (Illumina, Inc.) for the whole genome or in a ±1 kb region of the transcription start site (TSS) of each gene. Specifically, the ratio of probes for methylated sequences to the total of the probes for methylated sequences and probes for nonmethylated sequences (in the present specification, referred to as a “β value”) was determined. The β value is 0.00 when no methylation is found in all CpG sites, and is 1.00 when all CpG sites are methylated.

First, genes with β value >0.8 in the negative control RKO cells were selected. Genes with β value <0.5 on HO and β value >0.8 in the presence of treatment with 0.1% DMSO on H75 were further selected from the selected genes.

Gene Whose Methylation Level is Selectively Reduced by Im0

Then, genes with β value <0.65 in the Im0-treated cells of H75 and β value >0.8 in the Im3-treated cells of H75 were selected as genes whose methylation was selectively reduced by Im0. 6099 probes corresponded thereto for the whole genome, and 1110 genes (2135 probes) corresponded thereto for the TSS±1 kb region.

Gene Whose Methylation Level is Selectively Reduced by Im3

Also, genes with β value <0.65 in the Im3-treated cells of H75 and β value >0.8 in the Im-ON-treated cells of H75 were selected as genes whose methylation was selectively reduced by Im3. 1171 probes corresponded thereto for the whole genome, and 254 genes (450 probes) corresponded thereto for the TSS±1 kb region.

Gene Whose Methylation Level is Reduced by Im0 and Im3 in Common

Genes with β value <0.65 in the Im0-treated cells of H75 and β value <0.65 in the Im3-treated cells of H75 were selected as genes whose methylation level was reduced by Im0 and Im3 in common. 19 probes corresponded thereto for the whole genome, and 9 genes (19 probes) corresponded thereto for the TSS±1 kb region.

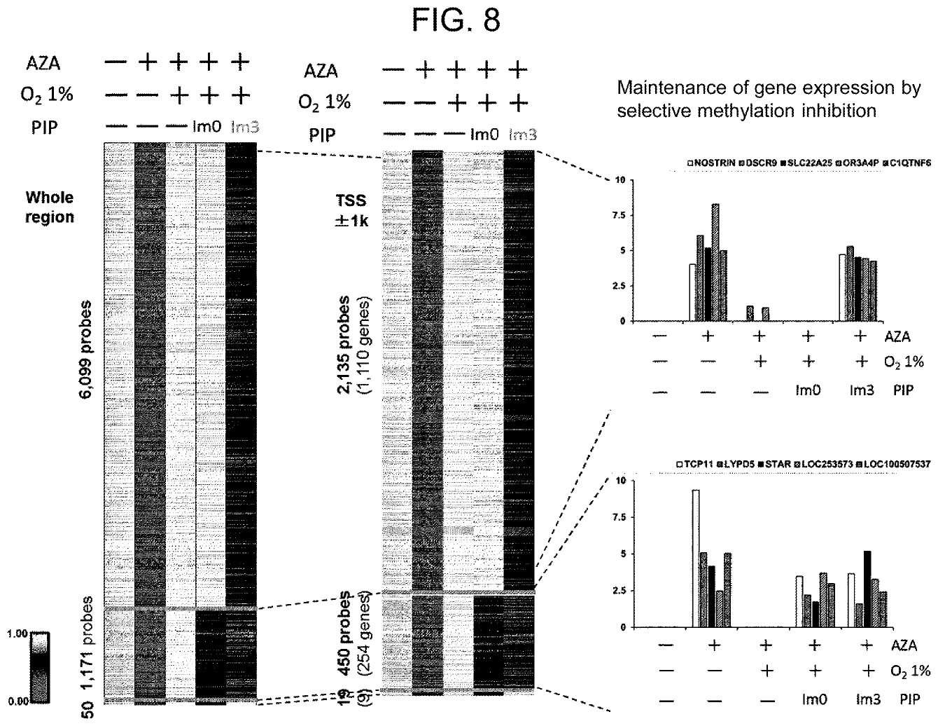

The results were as shown in FIG. 8.

As shown in FIG. 8, DNA is demethylated genome-wide by AZA treatment. By contrast, genome-wide methylation is induced by culture under hypoxic conditions. On the other hand, the methylation levels of some genes were drastically reduced in the genome of the Im0-treated cells. Also, the methylation levels of genes as a whole were drastically reduced in the genome of the Im3-treated cells.

Next, the expression levels of particular genes were analyzed. An RNA library was prepared using TruSeq Stranded mRNA Sample Prep Kit (Illumina, Inc.) according to the manufacturer's manual. Deep sequencing was performed using Illumina HiSeq 1500 or NextSeq 500 platform. FASTQ reads were mapped using TopHat, and transcripts were assembled using Cufflinks. gene expression was expressed as FPKM (fragments per kilobase of exon per million reads mapped). The results were as shown in the right graphs of FIG. 8.

As shown in the right graphs of FIG. 8, the gene expression of NOSTRIN, DSCR9, SLC22A25, OR3A4P and C1WTNF6 was selectively maintained by Im3. Also, the gene expression of TCP11, LYPDS, STAR, LOC253573 and LOC100507573 was maintained by Im0 and Im3. Thus, Im0 or Im3 was able to reduce the methylation level of a gene in a sequence-specific manner and to reduce maintain the normal gene expression level in cells. In conclusion, PIP comprising four monomer units remarkably controlled a gene expression level and a methylation level genome-wide.

### Example 6B: Development of Conjugate of Histone Modifying Enzyme Inhibitor and PIP

In this Example, whether gene expression that is not activated by a histone modifying enzyme inhibitor alone could be activated using a conjugate of PIP comprising four monomer units and the inhibitor, was examined.

The conjugate of the histone modifying enzyme inhibitor and PIP was obtained by linking compound 4 given below to the amino group at the amino terminus of Im0 or Im3. The conjugate of Im0 and the LSD1 inhibitor thus obtained is referred to as Im0-N, and the conjugate of Im3 and the LSD1 inhibitor thus obtained is referred to as Im3-N. The compound 4 is an analog of NCD38 and is a compound optimized for fusion with PIP and maintenance of LSD1 inhibitory activity.

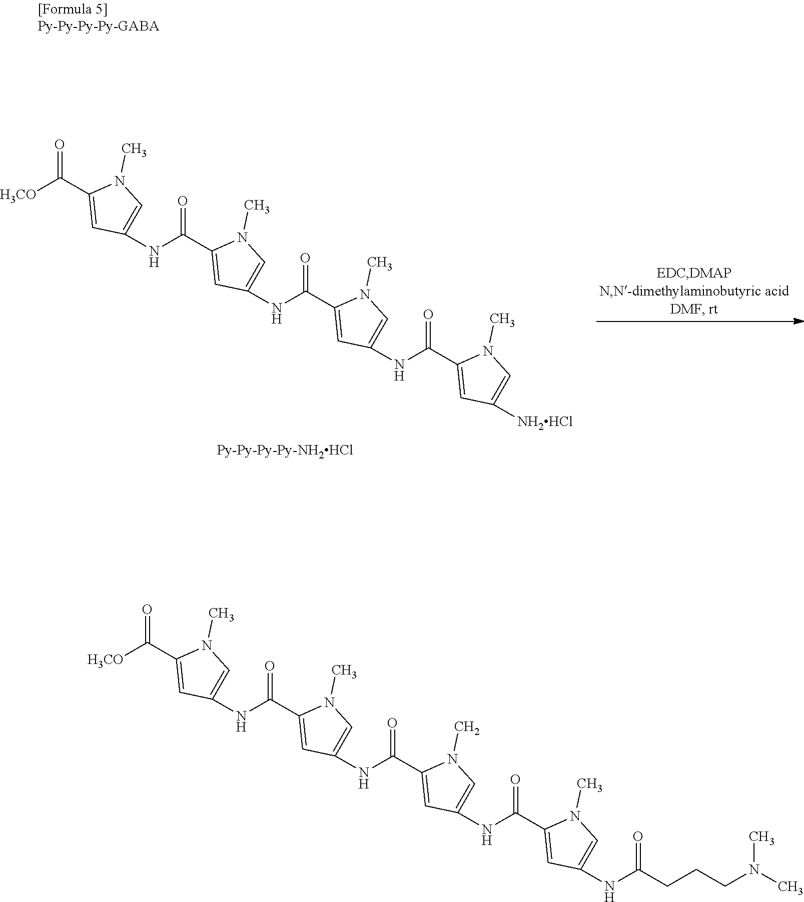

To a solution of Py-Py-Py-Py-NH₂.HCl (27.8 mg, 0.05 mmol) synthesized on the basis of the existing synthesis approach (J. Am. Chem. Soc. 1996, 118, 6141), 4-dimethylaminobutyric acid (16.8 mg, 0.1 mmol), and DMAP (12.2 mg, 0.1 mmol) in DNF (0.1 mL), EDC.HCl (19.1 mg, 0.1 mmol) was added, and the mixture was stirred at room temperature for 12 hours. A CHCl₃/i-PrOH (1/1) solution was added to the reaction solution, and the mixture was washed with a saturated aqueous solution of sodium bicarbonate and then dried over Na₂SO₄. The solvent was distilled off under reduced pressure, and then, the residue was purified by silica gel column chromatography (CHROMATOREX NH-DM1020, CHCl₃/MeOH=50/1 to 30/1) to obtain 17.0 mg (54% yield) of the product of interest.

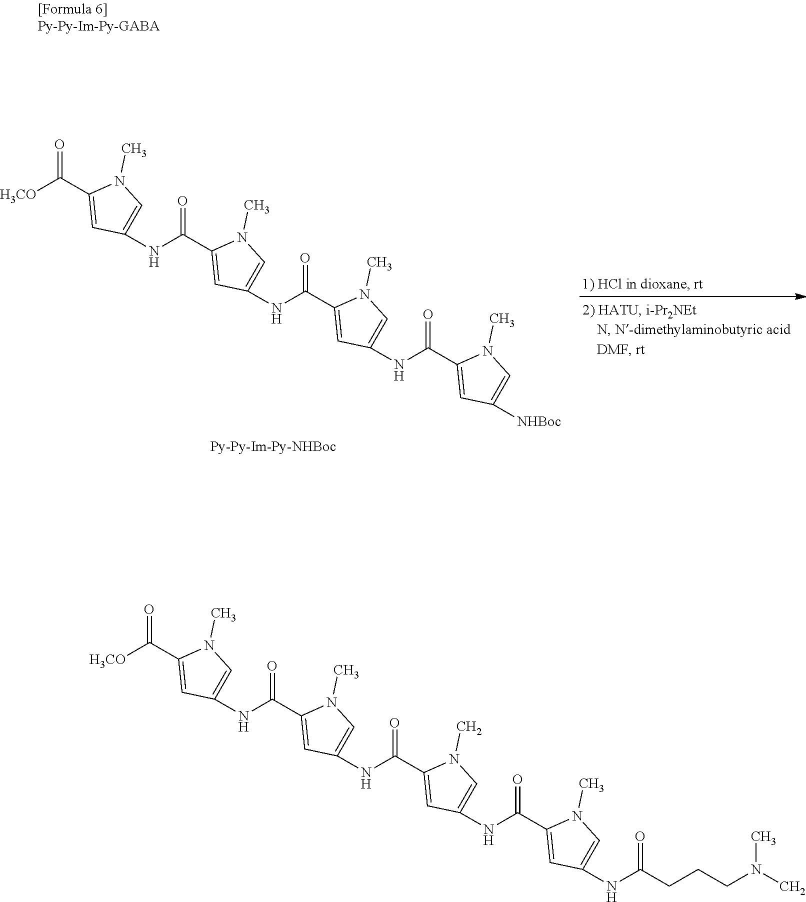 [SMILES: COC(=O)c1cc(NC(=O)c2cc(NC(=O)c3cc(NC(=O)c4cc(NC(=O)OC(C)(C)C)cn4C)cn3C)cn2C)cn1C ; COC(=O)c1cc(NC(=O)c2cc(NC(=O)c3cc(NC(=O)c4cc(NC(=O)CCCN(C)C)cn4C)cn3C)cn2C)cn1C]

To Py-Py-Im-Py-NHBoc (90 mg, 0.145 mmol) synthesized on the basis of the existing synthesis approach (J. Am. Chem. Soc. 1996, 118, 6141), a 4 N solution of HCl in dioxane (0.48 mL) was added, and the mixture was stirred at room temperature for 5 hours. The solvent was distilled off under reduced pressure, and then, the residue was washed with hexane to obtain 80.3 mg (99% yield) of amine hydrochloride.

4-Dimethylaminobutyric acid (16.8 mg, 0.1 mmol), HATU (38 mg, 0.1 mmol), and diisopropylethylamine (51 μL, 0.3 mmol) were dissolved in DMF (0.25 mL), and then, the solution was stirred at room temperature for 30 minutes. A solution of the amine hydrochloride (27.9 mg, 0.05 mmol) in DMF (0.25 mL) was added to the stirred solution at room temperature, and the mixture was stirred with the temperature unchanged for 16 hours. A 1 M aqueous KHSO₄ solution was added thereto, and the reaction solution was subjected to extraction with a CHCl₃/i-PrOH (1/1) solution. The organic phase was washed with water, a saturated aqueous solution of sodium bicarbonate, and saturated saline and then dried over Na₂SO₄. The solvent was distilled off under reduced pressure, and then, the residue was purified by silica gel column chromatography (CHROMATOREX NH-DM1020, CHCl₃/MeOH=50/1) to obtain 10.4 mg (33% yield) of the product of interest.

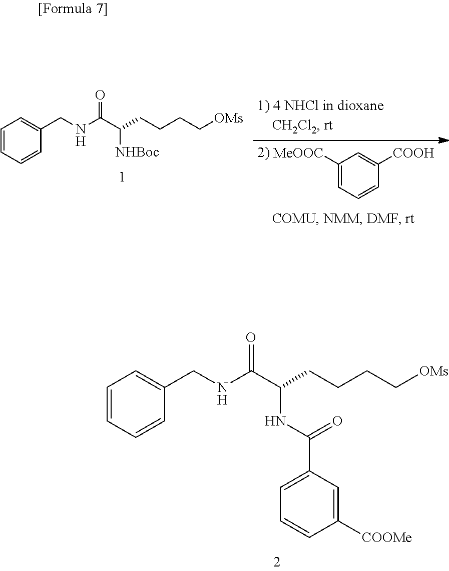 [SMILES: CC(C)(C)OC(=O)N[C@@H](CCCCOS(C)(=O)=O)C(=O)NCc1ccccc1 ; COC(=O)c1cccc(C(=O)O)c1 ; COC(=O)c1cccc(C(=O)N[C@@H](CCCCOS(C)(=O)=O)C(=O)NCc2ccccc2)c1]

To a solution of compound 1 (649.7 mg, 1.55 mmol) synthesized by the known approach (Angew. Chem. Int. Ed. 2013, 52, 8620) in CH₂Cl₂ (6.2 mL), 4 N HCl in dioxane (3.9 mL) was added at 0° C. The reaction solution was stirred at room temperature for 1 hour, and the solvent was distilled off under reduced pressure to obtain corresponding hydrochloride. To a solution of 3-(methoxycarbonyl)benzoic acid (279 mg 1.55 mmol), COMU (633.8 mg, 1.55 mmol), and N-methylmorpholine (0.68 mL, 6.2 mmol) in DMF (5 mL), a solution of the obtained hydrochloride in DMF (2.8 mL) was added at 0° C. 12 hours later, the reaction was terminated with a saturated aqueous solution of ammonium chloride, and ethyl acetate was added thereto. The mixed solution was washed with 1 N hydrochloric acid, a saturated aqueous solution of sodium bicarbonate, and saturated saline, and then, the organic phase was dried over sodium sulfate. The solvent was distilled off under reduced pressure, and then, the residue was recrystallized (ethyl acetate/hexane) to obtain compound 2 (480.9 mg, 65% yield).

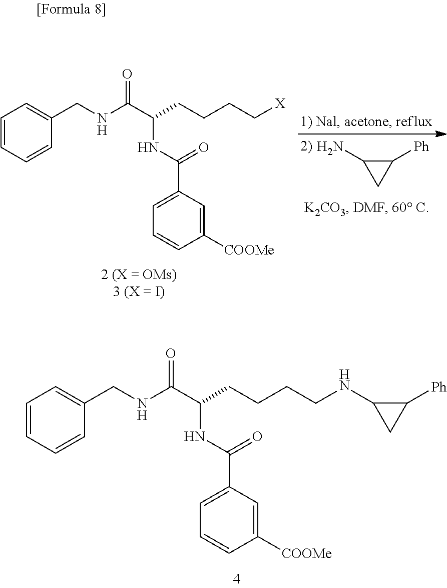

A suspension of compound 2 (206.7 mg, 0.43 mmol) and sodium iodide (1.2 g, 8 mmol) in acetone was heated to reflux for 18 hours. After temperature adjustment of the reaction solution to room temperature, ethyl acetate was added thereto, and the reaction solution was washed with saturated saline. The reaction solution was dried over sodium sulfate, and then, the solvent was distilled off under reduced pressure to obtain compound 3. This compound was used directly in the next reaction. A suspension of compound 3, PCPA hydrochloride (218.1 mg, 1.29 mmol), and potassium carbonate (355.8 mg, 2.58 mmol) in DMF (0.86 mL) was stirred at 60° C. for 48 hours. After temperature adjustment of the reaction solution to room temperature, ethyl acetate was added thereto, and the mixed solution was washed with a saturated aqueous solution of sodium bicarbonate and saturated saline and dried over sodium sulfate. The solvent was distilled off under reduced pressure, and then, the residue was purified by silica gel column chromatography (SiO₂, AcOEt, then CHCl₃/MeOH=20/1 to 10/1) to obtain 191.5 mg (86% yield) of compound 4.

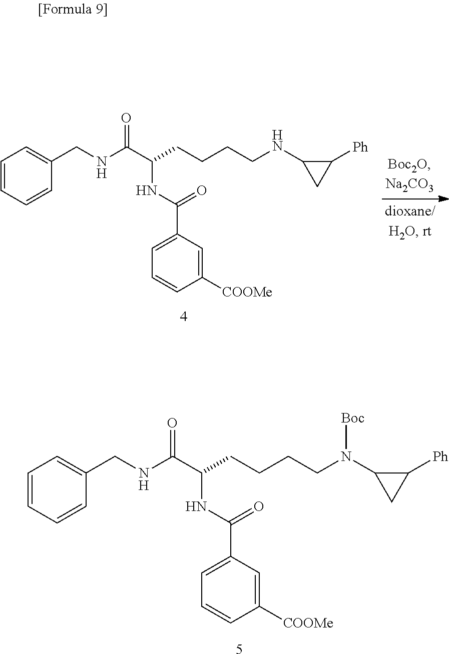

Compound 4 (144.3 mg, 0.28 mmol) was stirred in a mixed solvent of dioxane (1.1 mL) and a 10% aqueous sodium carbonate solution (0.3 mL), and Boc₂O (368.3 mg, 1.68 mmol) was added thereto at room temperature. After stirring for 2.5 hours, ethyl acetate was added thereto, and the solution was washed with saturated saline and then dried over sodium sulfate. The solvent was distilled off under reduced pressure, and then, the residue was purified by silica gel column chromatography (SiO₂, hexane/AcOEt=2/1 to 0/1) to obtain 91.6 mg (53% yield) of compound 5.

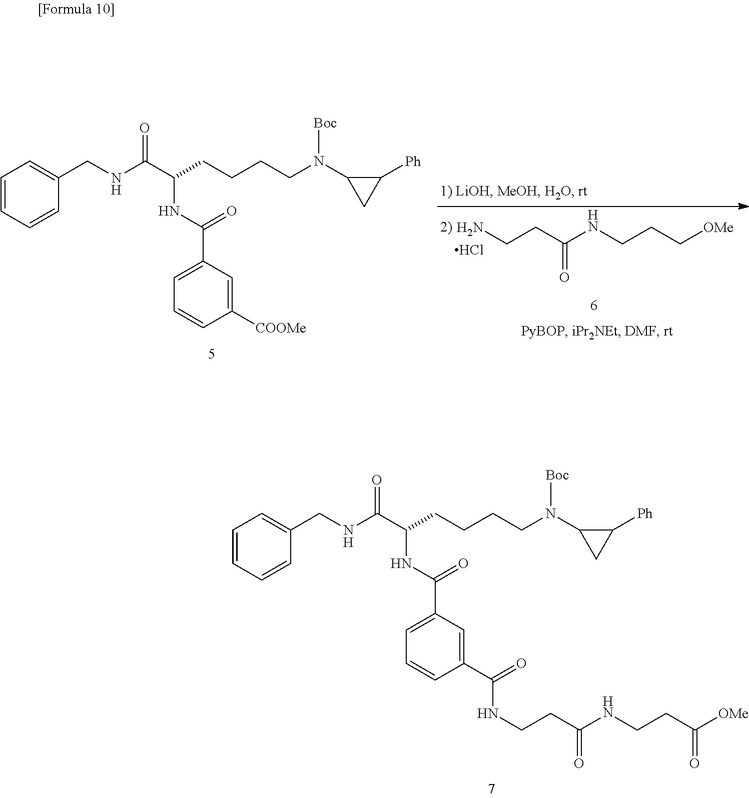

To a solution of compound 5 (91.6 mg, 0.15 mmol) in methanol (0.75 mL), a 1 M aqueous LiOH solution (0.3 mL) was added at 0° C. 12 hours later, ethyl acetate was added thereto, and the reaction solution was rendered acidic with a 10% aqueous citric acid solution. After extraction with chloroform, the solution was dried over sodium sulfate. The solvent was distilled off under reduced pressure, and then, the residue was used in the next reaction. To a solution of the obtained residue, PyBOP (135.2 mg, 0.26 mmol), and known compound 6 (Chem. Eur. J. 2013, 19, 15822) (55.6 mg, 0.26 mmol) in DMF (0.65 mL), iPr₂NEt (0.09 mL, 0.52 mmol) was added at 0° C. After stirring at room temperature for 4 hours, ethyl acetate was added thereto. The mixture was washed with 1 N hydrochloric acid, a saturated aqueous solution of sodium bicarbonate, and saturated saline in this order and then dried over sodium sulfate. The solvent was distilled off under reduced pressure, and then, the residue was purified by silica gel column chromatography (SiO₂, CHCl₃/MeOH=20/1 to 10/1) to obtain 95.6 mg (84% yield) of compound 7.

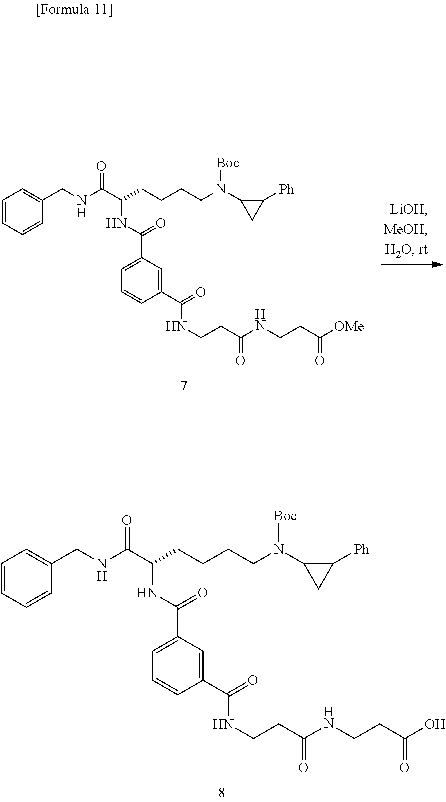 [SMILES: COC(=O)CCNC(=O)CCNC(=O)c1cccc(C(=O)N[C@@H](CCCCN(C(=O)OC(C)(C)C)C2CC2c2ccccc2)C(=O)NCc2ccccc2)c1 ; CC(C)(C)OC(=O)N(CCCC[C@H](NC(=O)c1cccc(C(=O)NCCC(=O)NCCC(=O)O)c1)C(=O)NCc1ccccc1)C1CC1c1ccccc1]

To a solution of compound 7 (52.5 mg, 0.0695 mmol) in methanol (0.7 mL), a 1 M aqueous LiOH solution (0.14 mL) was added at 0° C. After stirring at room temperature for 12 hours, the mixture was subjected to back extraction with ethyl acetate. The aqueous phase was rendered acidic with a 10% aqueous citric acid solution, followed by extraction with chloroform. The organic phase was dried over sodium sulfate. Then, the solvent was distilled off under reduced pressure, and the residue was purified by silica gel column chromatography (SiO₂, CHCl₃/MeCH=10/1 to 0/1) to obtain 24.5 mg (48% yield) of compound 8.

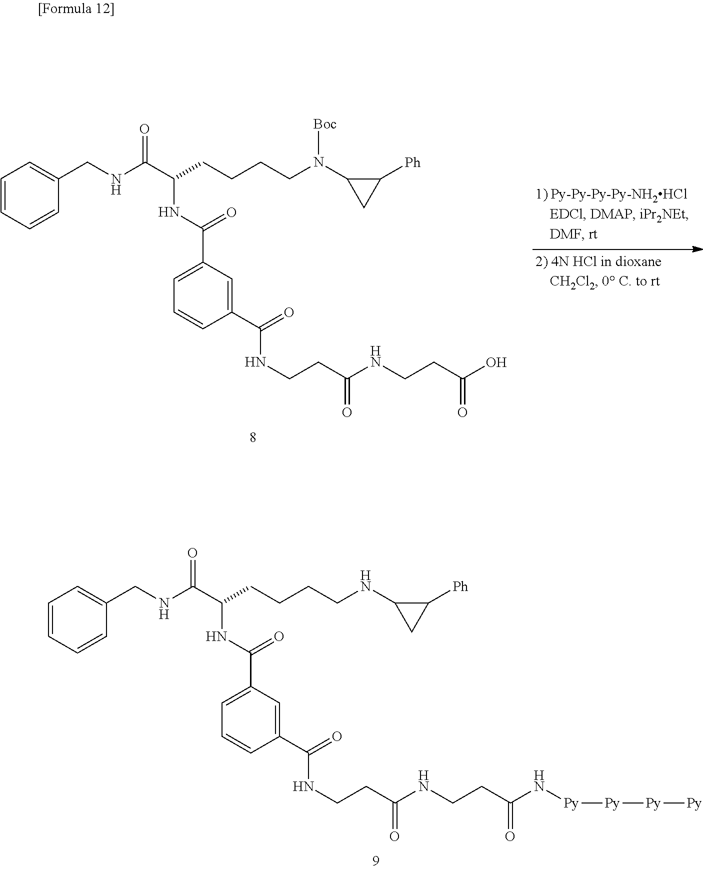

To a solution of compound 8 (35.2 mg, 0.0048 mmol), DMAP (12 mg, 0.096 mmol), and EDCI (18.4 mg, 0.096 mmol) in DMF (0.96 mL), Py-Py-Py-Py-NH₂.HCl (26.7 mg, 0.048 mmol) and iPr₂NEt (32 μL, 0.192 mmol) were added at 0° C. After stirring at room temperature for 15 hours, ethyl acetate was added thereto, and the mixture was washed with 1 N hydrochloric acid, a saturated aqueous solution of sodium bicarbonate, and saturated saline in this order. The organic phase was dried over sodium sulfate. Then, the solvent was distilled off under reduced pressure, and the residue was purified by silica gel column chromatography (CHROMATOREX NH-DM1020, CHCl₃/MeOH=20/1) to obtain 15.7 mg of a crude product of a condensate. 15.7 mg of the obtained crude product was dissolved in methylene chloride (1.2 mL), and 4 N HCl in dioxane (0.12 mL) was added to the solution at 0° C. After stirring at room temperature for 2 hours, water was added thereto, and the mixture was subjected to back extraction with methylene chloride. The obtained aqueous phase was rendered basic with a 1 M LiOH solution, followed by extraction with chloroform. The organic phase was dried over sodium sulfate. Then, the solvent was distilled off under reduced pressure, and the residue was purified by silica gel column chromatography (CHROMATOREX NH-DM1020, CHCl₃/MeOH=20/1 to 10/1) to obtain 1.8 mg (3% yield) of product 9 (i.e., Im0-N).

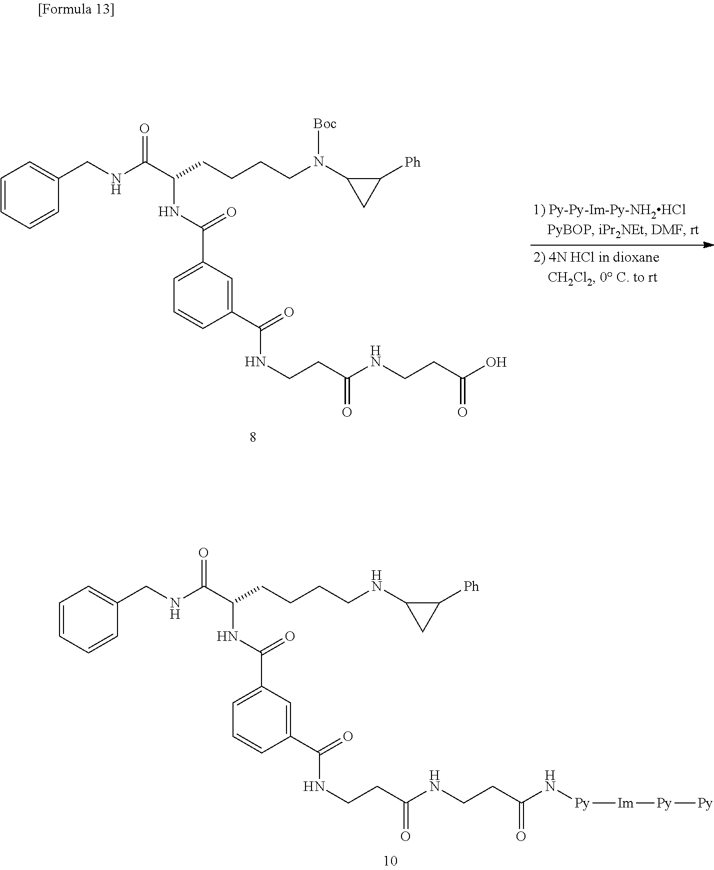

To a solution of compound 8 (19.5 mg, 0.026 mmol) and PyBOP (19.5 mg, 0.039 mmol) in DNF (0.52 mL), Py-Py-Im-Py-NH₂.HCl (14.7 mg, 0.026 mmol) and iPr₂NEt (18 μL, 0.104 mmol) were added at 0° C. After stirring at room temperature for 15 hours, ethyl acetate was added thereto, and the mixture was washed with a 1 N aqueous potassium bisulfate solution, a saturated aqueous solution of sodium bicarbonate, and saturated saline in this order. The organic phase was dried over sodium sulfate. Then, the solvent was distilled off under reduced pressure, and the residue was purified by silica eel column chromatography (CHROMATOREX NH-DM1020, CHCl₃/MeOH=20/1) to obtain 26.5 mg of a crude product of a condensate. 26.5 mg of the obtained crude product was dissolved in methylene chloride (0.42 mL), and 4 N HCl in dioxane (0.05 mL) was added to the solution at 0° C. After stirring at room temperature for 4 hours, water was added thereto, and the mixture was subjected to back extraction with methylene chloride. The obtained aqueous phase was rendered basic with a 1 M LiOH solution, followed by extraction with chloroform. The organic phase was dried over sodium sulfate. Then, the solvent was distilled off under reduced pressure, and the residue was purified by silica gel column chromatography (CHROMATOREX NH-DM1020, CHCl₃/MeOH=20/1) to obtain 4.9 mg (16% yield) of product 10 (i.e., Im3-N).

The large intestine cancer cell line RKO cells were seeded at a density of 1×10⁵ cells over a 6 cm dish and incubated for 24 hours. Subsequently, the culture solution was replaced with a culture solution containing 2 μM NCD38 and Im0-N or Im3-N. The cultured cells were exposed to the drug over 30 days. During this operation, the medium containing the compound was replaced every 5 days. The cells were subcultured at a density of 2×10⁵ cells over a 6 cm dish when becoming almost confluent. The cells were recovered on the 30th day.

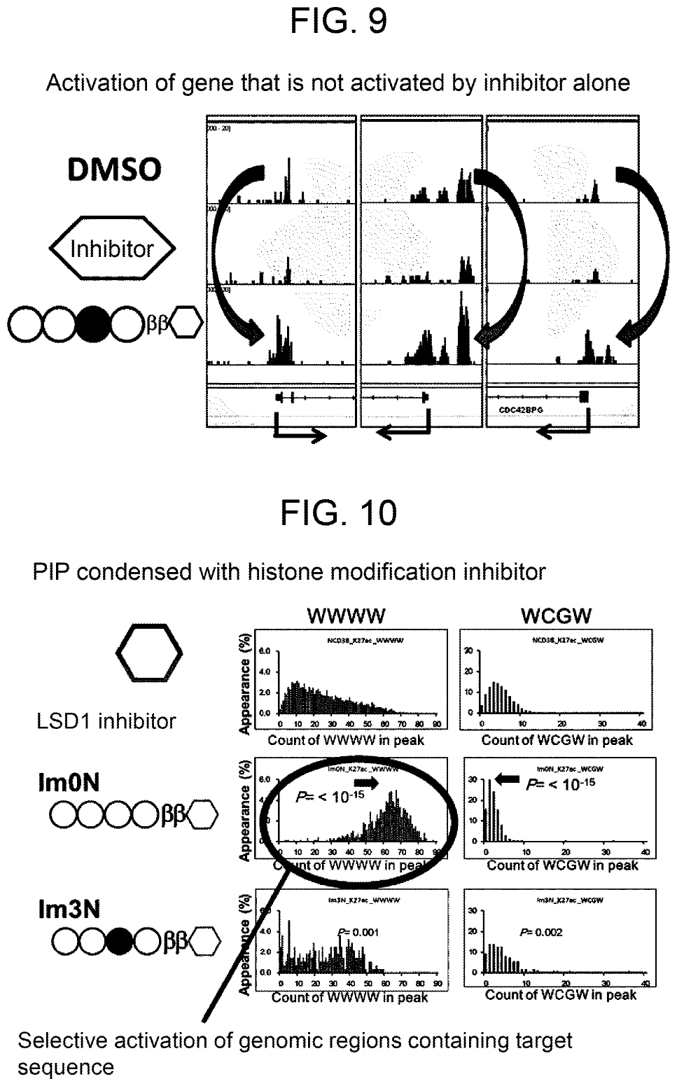

Next, the recovered cells were fixed in 1% formaldehyde. Then, fragmented chromatin DNA was prepared by ultrasonication. For the fragmented chromatin, a region having increased histone methylation, which is one of the gene activation markers, was identified by ChIP-Seq using an antibody against histone methylation (H3K4me3). As a result, as shown in FIG. 9, no large variation in histone methylation was found in a plurality of gene promoter regions by the inhibitor treatment, whereas a phenomenon in which histone methylation was increased was found in these regions by the Im3-N treatment. This suggested that the PIP-LSD1 inhibitor selectively induces histone activation.

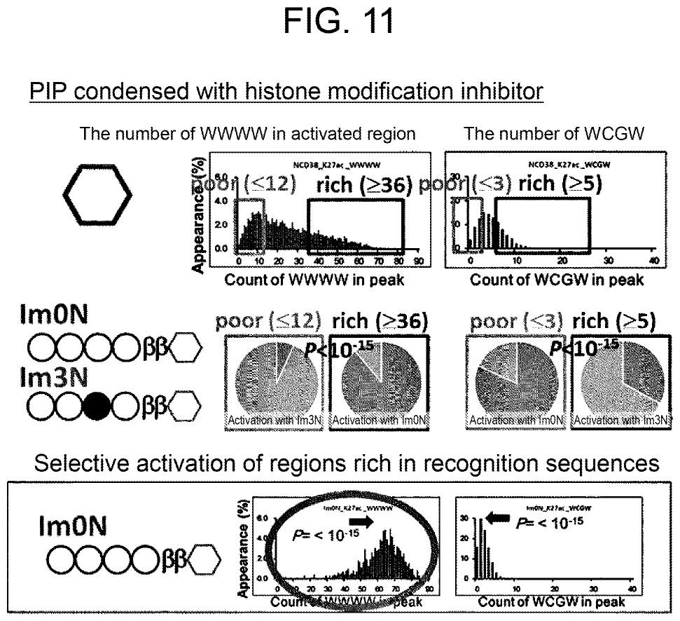

The sequence specificity of delivery of the histone modifying enzyme inhibitor onto the genome by PIP was confirmed from the viewpoint of change in histone acetylation, which is another histone activation marker. The cells were treated with Im0-N or Im3-N by the method described in Example 5B, and a histone activation region was identified by ChIP-Seq using an antibody against histone acetylation (H3K27Ac). The results were as shown in FIGS. 10 and 11. As shown in FIGS. 10 and 11, the number of target sequences having WWWW was found significantly and remarkably larger in the Im0-N-activated histone region, as compared with the treatment system with NCD38 alone. Also, the number of target sequences having WCGW was found significantly larger in the Im3-N-activated histone region.

The results mentioned above demonstrated that at least four monomer units of PIP suffice sequence-dependent binding to genomic DNA. The results also demonstrated that PIP can deliver an additional molecule linked thereto to genomic DNA in a sequence-dependent manner.

1. A method for inhibiting repression of gene expression by DNA methylation in a living subject, the method comprising:
administering to the subject pyrrole imidazole polyamide,
wherein the pyrrole imidazole polyamide binds in a sequence-specific manner to a minor groove of promoter region DNA of a gene.

2. (canceled)

3. The method according to claim 1, wherein the DNA methylation is DNA methylation that occurs intracellularly after DNA demethylation treatment.

4. The method according to claim 3, wherein the DNA methylation is DNA methylation that occurs intracellularly after DNA demethylation treatment and inhibition treatment of histone deacetylation.

5. The method according to claim 3, wherein the DNA demethylation treatment is performed by 5-aza-2′-deoxycytidine (AZA) treatment.

6. The method according to claim 4, wherein the inhibition treatment of histone deacetylation is performed by trichostatin A treatment.

7. The method according to claim 1, wherein the gene is a cancer suppressor gene.

8. The method according to claim 7, wherein the cancer suppressor gene is MLH1 gene.

9. A method for treating a cancer in a subject with the cancer, preventing onset of a cancer or reducing a risk of developing cancer in a subject, the method comprising:
administering to the subject in need thereof, pyrrole imidazole polyamide binding in a sequence-specific manner to a promoter region of a cancer suppressor gene having methylated promoter region DNA, thereby inhibiting transcriptional repression of the cancer suppressor gene.

10. The method according to claim 9,
wherein the cancer is a cancer selected from the group consisting of large intestine cancer, stomach cancer, gastric pit-type stomach cancer, esophageal cancer, head and neck squamous cell carcinoma, non-small cell lung cancer and colorectal cancer, and
the cancer suppressor gene is MLH1 gene.

11. The method according to claim 9, wherein the administering further comprises administering a DNA demethylating agent and a histone deacetylation inhibitor.

12. The method according to claim 9, wherein the subject has increased methylation of MLH1 gene.

13. A method for inhibiting DNA methylation in target DNA or the vicinity thereof in a living subject, the method comprising:
administering to the subject pyrrole imidazole polyamide, wherein the pyrrole imidazole polyamide binds in a sequence-specific manner to the target DNA.
